(19) United States
(12) Patent Application Publication
(10) Pub. No.: US 2026/0078376 A1
(43) Pub. Date: Mar. 19, 2026

(54) OLIGONUCLEOTIDES FOR THE TREATMENT OF BREAST CANCER

(71) Applicant: CORE BIOTHERAPEUTICS, INC., Ladera Ranch, CA (US)

(72) Inventors: Michael KLUKINOV, Pittsburg, CA (US); David C. YEOMANS, Sunnyvale, CA (US); Julien MAMET, Hayward, CA (US)

(21) Appl. No.: 19/106,594

(22) PCT Filed: Aug. 25, 2023
(86) PCT No.: PCT/US2023/072946

§ 371 Date: Feb. 25, 2025

Related U.S. Application Data
(60) Provisional application No. 63/373,658, filed on Aug. 26, 2022.
(60) Provisional application No. 63/477,753, filed on Dec. 29, 2022.

(51) Int. Cl. C12N 15/113; A61K 31/712; A61K 31/713; A61P 35/00

(52) U.S. Cl. CPC C12N 15/1135; A61K 31/712; A61K 31/713; A61P 35/00; C12N 15/1136; C12N 2310/13; C12N 2310/322

(57) ABSTRACT

Provided are therapeutic agents such as double-stranded nucleic acids, termed oligonucleotide decoys, pharmaceutical compositions comprising the same, and related methods of modulating breast cancer signaling, for instance, to treat breast cancer.

### CROSS-REFERENCE TO RELATED APPLICATIONS

The present application claims priority to U.S. Provisional Application No. 63/373,658, filed Aug. 26, 2022 and U.S. Provisional Application No. 63/477,753, filed Dec. 29, 2022, the entire contents of each of which are hereby incorporated by reference in their entirety for all purposes.

### SEQUENCE LISTING

The contents of the electronic sequence listing (NEBI_001_01WO_SeqList_ST26.xml; Size: 5,967,445 bytes; and Date of Creation: Aug. 25, 2023) are herein incorporated by reference in its entirety.

### FIELD OF THE INVENTION

The present invention relates to therapeutic agents such as double-stranded nucleic acids, termed oligonucleotide decoys, pharmaceutical compositions comprising the same, and related methods of treating breast cancer.

### BACKGROUND

Cancer is a large group of diseases that can start in almost any organ or tissue of the body when abnormal cells grow uncontrollably, go beyond their usual boundaries to invade adjoining parts of the body and/or spread to other organs. The latter process is called metastasizing and is a major cause of death from cancer. A neoplasm and malignant tumor are other common names for cancer.

Cancer is the second leading cause of death globally, accounting for an estimated 9.6 million deaths, or one in six deaths, in 2018.

Breast cancer, is amongst the most common cancer type among women but can also affect men. There are many types of breast cancer, and many different ways to describe them. The type of breast cancer is determined by the specific cells in the breast that are affected. Most breast cancers are carcinomas, which are tumors that start in the epithelial cells that line organs and tissues throughout the body. When carcinomas form in the breast, they are usually a more specific type called adenocarcinoma, which starts in cells in the ducts (the milk ducts) or the lobules (milk-producing glands).

The type of breast cancer can also refer to whether the cancer has spread or not. In situ breast cancer (ductal carcinoma in situ, or DCIS) is a cancer that starts in a milk duct and has not grown into the rest of the breast tissue. The term invasive or infiltrating breast cancer is used to describe any type of breast cancer that has spread or invaded into the surrounding breast tissue. Some invasive breast cancers have special features or develop in different ways that affect their treatment and outlook. These cancers are less common but can be more serious than other types of breast cancer. They include: Triple-negative breast cancer (TNBC) accounts for about 10-15% of all breast cancers. The term triple-negative breast cancer refers to the fact that the cancer cells don't have estrogen or progesterone receptors and also don't make too much of the protein called HER2.

Inflammatory breast cancer (IBC) is rare and accounts for 1-5% of all breast cancers. Although it is often a type of invasive ductal carcinoma, it differs from other types of breast cancer in its symptoms, outlook, and treatment. IBC has symptoms of inflammation like swelling and redness, but infection or injury do not cause IBC or the symptoms IBC symptoms are caused by cancer cells blocking lymph vessels in the skin causing the breast to look “inflamed.”

Triple-negative breast cancer differs from other types of invasive breast cancer in that they grow and spread faster, have limited treatment options, and a worse prognosis.

Other types can include Paget disease of the breast, Angiosarcoma, and phyllode tumors.

Breast tumors can also be classified by the type of receptor or market that they express or not, including but not limited to estrogen receptor (ER+), progesterone receptor (PR+), Her2 receptor (HER2+), and androgen receptors (AR+).

Current treatments include surgery, chemotherapy, radiation therapy, targeted therapy, immunotherapy, stem cell or bone marrow transplant, and hormone therapy. While treatments can be efficacious in some tumors and can improve the disease state and/or patient survival, they are ineffective in a range of tumor types. The toxicity of those treatments is also significant, debilitating, and can in cases even prevent the use of the treatments.

Thus, there is a need in the art for improved breast cancer treatment methods.

### SUMMARY OF THE INVENTION

The present disclosure provides double-stranded nucleic acids termed oligonucleotide decoys and pharmaceutical compositions comprising the same, and related methods of modulating and treating breast cancer.

In embodiments, the present disclosure provides an oligonucleotide decoy, comprising at least two transcription factor binding sites, wherein each transcription factor binding site binds to a transcription factor selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors. In embodiments, the oligonucleotide decoy is about 10 to about 100 base pairs in length. In embodiments, the oligonucleotide decoy binds 2 to 40 transcription factors. In embodiments, the oligonucleotide decoy comprises a first transcription factor binding site and a second transcription factor binding site, and wherein the first and the second transcription binding sites overlap. In embodiments, the oligonucleotide decoy further comprises a third transcription factor binding site, wherein the first, second, and third transcription factor binding sites overlap. In embodiments, the oligonucleotide decoy comprises 3-40 transcription factor binding sites that overlap. In embodiments, the oligonucleotide decoy comprises transcription factor binding sites that do not overlap. In embodiments, the oligonucleotide decoy is 12, 17, 18, 29, 41, 44, or 57-nucleotide long.

In embodiments, the present disclosure provides a nucleotide sequence that is at least 80%, at least 85%, at least 90%, at least 95%, or 100% identical to a sequence selected from the group consisting of SEQ ID NO: 1 to SEQ ID NO: 48. In embodiments, the oligonucleotide decoy binds to one or more transcription factors selected from the group consisting of: TCF7L1 and GTF2IRD1.

In embodiments, the present disclosure provides a nucleotide sequence that is at least 80%, at least 85%, at least 90%, at least 95%, or 100% identical to a sequence selected from the group consisting of SEQ ID NO: 49 to SEQ ID NO: 64. In embodiments, the oligonucleotide decoy binds to one or more transcription factors selected from the group consisting of: FOXC1, FOXM1, GTF2IRD1, and TCF7L1.

In embodiments, the present disclosure provides a nucleotide sequence that is at least 80%, at least 85%, at least 90%, at least 95%, or 100% identical to a sequence selected from the group consisting of SEQ ID NO: 3761 to SEQ ID NO: 3836. In embodiments, the oligonucleotide decoy binds to one or more transcription factors selected from the group consisting of: BCL11A, HMGA1, HOXB5, and MAF.

In embodiments, the present disclosure provides a nucleotide sequence that is at least 80%, at least 85%, at least 90%, at least 95%, or 100% identical to a sequence selected from the group consisting of SEQ ID NO: 3837 to SEQ ID NO: 3868. In embodiments, the oligonucleotide decoy binds to one or more transcription factors selected from the group consisting of: BCL11A, HMGA1, HOXB5, and MAF.

In embodiments, the present disclosure provides a nucleotide sequence that is at least 80%, at least 85%, at least 90%, at least 95%, or 100% identical to a sequence selected from the group consisting of SEQ ID NO: 65 to SEQ ID NO: 176, and SEQ ID NO: 4593. In embodiments, the oligonucleotide decoy binds to one or more transcription factors selected from the group consisting of: E2F3, E2F5, FOXC1, FOXM1, GTF2IRD1, LYL1, TCF4, TCF7L1, TGIF2, and ZEB2.

In embodiments, the present disclosure provides a nucleotide sequence that is at least 80%, at least 85%, at least 90%, at least 95%, or 100% identical to a sequence selected from the group consisting of SEQ ID NO: 177 to SEQ ID NO: 3760. In embodiments, the oligonucleotide decoy binds to one or more transcription factors selected from the group consisting of: CXXC5, FOXC1. FOXM1, MAX, NFIL3, TCF4, XBP1, YBX1, YY1, and ZEB2.

In embodiments, the present disclosure provides a nucleotide sequence that is at least 80%, at least 85%, at least 90%, at least 95%, or 100% identical to a sequence selected from the group consisting of SEQ ID NO: 3869 to SEQ ID NO: 4540. In embodiments, the oligonucleotide decoy binds to one or more transcription factors selected from the group consisting of: BCL11A, E2F3, E2F5, FOXC1, FOXM1, GTF2IRD1, HMGA1, HOXB5, LYL1, MAF, TCF4, TCF7L1, TGIF2, and ZEB2.

In embodiments, the oligonucleotide decoy binds to one or more transcription factors selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

In embodiments, the present disclosure provides a nucleotide sequence that is at least 80%, at least 85%, at least 90%, at least 95%, or 100% identical to a sequence selected from the group consisting of SEQ ID NO: 88, SEQ ID NO: 2225, SEQ ID NO: 4593, SEQ ID NO: 4594, SEQ ID NO: 4595, SEQ ID NO: 4596, SEQ ID NO:4547, and SEQ ID NO: 4598.

In embodiments, the oligonucleotide decoy is a double-stranded nucleic acid.

In embodiments, the present disclosure provides a pharmaceutical composition, comprising an oligonucleotide decoy or population of oligonucleotide decoy as described herein, and a pharmaceutically acceptable carrier.

In embodiments, the present disclosure provides a kit, comprising an oligonucleotide decoy or population of oligonucleotide decoys as described herein, and optionally an instruction for using said oligonucleotide decoy.

In embodiments, the present disclosure provides a method for modulating transcription of a gene present in a cell involved in breast cancer signaling, comprising administering to the cell an effective amount of an oligonucleotide decoy as described herein. In embodiments, the present disclosure provides a method for modulating breast cancer signaling in a cell, comprising administering to the cell an effective amount of an oligonucleotide decoy as described herein. In embodiments, the present disclosure provides a method for treating breast cancer in a subject in need thereof, comprising administering to the subject a therapeutically effective amount of an oligonucleotide decoy as described herein. In embodiments, the breast cancer is a triple negative breast cancer. In embodiments, the breast cancer is an ER+ or PR+ breast cancer. In embodiments, the breast cancer is an HER2+ breast cancer.

In embodiments, the present disclosure provides a method for modulating breast cancer signaling in a cell, comprising administering to the cell a therapeutically effective amount of a therapeutic agent, wherein the therapeutic agent inhibits binding of a transcription factor to its transcription factor binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

In embodiments, the present disclosure provides a method for treating breast cancer in a subject in need thereof, comprising administering to the subject a therapeutically effective amount of a therapeutic agent, wherein the therapeutic agent inhibits binding of a transcription factor to its transcription binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

In embodiments, the present disclosure provides therapeutic agents, such as oligonucleotides, which inhibit the binding of at least one of the following transcription factors: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors to its (their) endogenous transcription factor binding site(s), pharmaceutical compositions comprising such agents, and related methods of modulating signaling, for example, to treat breast tumor and limit its progression, growth, migration, invasiveness, and/or metastasis and/or treat secondary metastatic tumors and/or recurrence, from breast cancer stem cells, in a subject in need thereof. In some embodiments, the therapeutic agents are double-stranded oligonucleotides (e.g., oligonucleotide decoys), which comprise one or more transcription factor binding sites that bind to at least one of the following transcription factors: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s).

Embodiments of the present invention therefore include oligonucleotide decoys comprising one or more transcription factor binding sites, wherein the one or more transcription factor binding sites bind to a transcription factor selected from the group consisting of: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s).

In some embodiments, the one or more transcription factor binding sites bind to one or more transcription factors (1, 2, 3, 4, 5, etc.), selected from one or more of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s).

In particular embodiments, the oligonucleotide decoys comprise a combination of at least two transcription factor binding sites, wherein each transcription factor binding site binds to a transcription factor selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s). In particular embodiments, each transcription factor binding site binds to a different transcription factor consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s).

In some embodiments, the oligonucleotide decoy is about 10 to about 200 base pairs in length.

In particular embodiments, the oligonucleotide decoy comprises transcription factor binding sites for different transcription factors that overlap. In particular embodiments, the oligonucleotide decoy comprises a transcription factor binding sites that are in opposite direction.

In certain embodiments, the oligonucleotide decoy has a first transcription factor binding site, and up to 1 other transcription factor binding sites. In specific embodiments, binding site binds to TCF7L1 and/or GTF2IRD1 and/or transcription factors related to those.

In certain embodiments, the oligonucleotide decoy has a first transcription factor binding site, and up to 3 other transcription factor binding sites. In specific embodiments, binding site binds to FOXC1 and/or FOXM1 and/or TCF7L1 and/or GTF2IRD1 and/or transcription factors related to those.

In certain embodiments, the oligonucleotide decoy has a first transcription factor binding site, an up to 1, 2, 3, 4, 5, 6, 7 or 8 other transcription factor binding sites. In specific embodiments, binding site binds to YBX1 and/or YY1, and/or ZEB2 and/or FOXM1 and/or FOXC1 and/or MAX and/or XBP1 and/or CXXC5 and/or NFIL3 and/or transcription factors related to those.

In certain embodiments, the oligonucleotide decoy has a first transcription factor binding site, an up to 1, 2, 3, 4, 5, 6, 7, 8, 9 or 10 other transcription factor binding sites. In specific embodiments, binding site binds to ZEB2 and/or FOXM1, and/or TCF4 and/or MAF and/or TCF7L1 and/or TGIF2 and/or LYL1 and/or GTF2IRD1 and/or FOXC1 and/or E2F3 and/or E2F5 and/or transcription factors related to those.

In certain embodiments, the oligonucleotide decoy has a first transcription factor binding site, an up to an up to 1, 2, 3, 4, 5, 6, 7, 8, 9 or 10 other transcription factor binding sites. In specific embodiments, binding site binds to YBX1 and/or YY1, and/or ZEB2 and/or FOXM1 and/or FOXC1 and/or TCF7L1 and/or TGIF2 and/or LYL1 and/or GTF2IRD1, and/or E2F3, and/or E2/F5 and/or transcription factors related to those.

In certain embodiments, the oligonucleotide decoy has a first transcription factor binding site, an up to 1, 2, 3, 4, 5, 6, 7, 8, 9, 10, 11, 12 or 13 other transcription factor binding sites. In specific embodiments, binding site binds to ZEB2 and/or FOXM1, and/or TCF4 and/or MAF and/or TCF7L1 and/or TGIF2 and/or LYL1 and/or GTF2IRD1 and/or FOXC1 and/or E2F3 and/or E2F5 and/or BCL11A, and/or HOXB5, and/or HMGA1 and/or transcription factors related to those.

In certain embodiments, the oligonucleotide decoy has a first transcription factor binding site, an up to 1, 2, 3, 4, 5, 6, 7, 8, 9, 10, 11, 12, 13, 14, 15, 16, 17, 18 or 19 other transcription factor binding sites. In specific embodiments, binding site binds to ZEB2 and/or FOXM1, and/or TCF4 and/or MAF and/or TCF7L1 and/or TGIF2 and/or LYL1 and/or GTF2IRD1 and/or FOXC1 and/or E2F3 and/or E2F5 and/or BCL11A, and/or HOXB5, and/or HMGA1 and/or YBX1, and/or YY1 and/or CXXC5, and/or NFIL3, and/or MAX, and/or XBP1 and/or transcription factors related to those.

In certain embodiments, the oligonucleotide decoy has a first transcription factor binding site, more than 20 other transcription factor binding sites In specific embodiments, binding site binds to any of combination of t transcription factors from the following list: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

Certain embodiments relate to one or more population(s) of the oligonucleotide decoys described herein, wherein the population of oligonucleotide decoys provide transcription factor binding sites to address combinations of multiple transcription factors that present the potential to treat multiple types of breast cancers (e.g., slow or stop tumor growth, reduce tumor size, inhibit tumor aggressiveness and tissue invasion and/or prevent metastasis).

Certain embodiments relate to one or more population(s) of the oligonucleotide decoys described herein, wherein the population of oligonucleotide decoys provide transcription factor binding sites to address combinations of multiple transcription factors that present the potential to block normal or stem cell transformation into cancer cells, cancer cell proliferation, favor cancer cell death (e.g. apoptosis), cancer cell migration, cancer cell invasion, epithelial-mesenchymal transition and/or metastasis.

In some embodiments, the therapeutic agent provides unique binding sites compositions and position that allow for binding more than one and up to 40 or more transcription factors at a time.

In some embodiments, the oligonucleotide decoy comprises a sequence represented Table A.

Also included are pharmaceutical compositions comprising an oligonucleotide decoy or population of decoys described herein and a pharmaceutically acceptable carrier. In certain embodiments, the oligonucleotide decoys are provided as salts, hydrates, solvates, or N-oxides derivatives.

Some embodiments include one or more kits comprising an oligonucleotide decoy or population of decoys described herein, optionally an instruction for using the oligonucleotide decoy(s).

Also included are methods for modulating the transcription of a gene present in a cell involved in cancer signaling comprising administering to the cell an effective amount of an oligonucleotide decoy or pharmaceutical composition described herein.

Also included are methods for modulating cancer signaling in a cell comprising administering to the cell an effective amount of an oligonucleotide decoy or pharmaceutical composition described herein.

Certain embodiments include methods for treating cancer and preventing relapse and/or metastasis in a subject comprising administering to the subject a therapeutically effective amount of an oligonucleotide decoy or pharmaceutical composition described herein. In some embodiments, the tumor is a triple negative breast tumor (i.e., negative for estrogen receptor, progesterone receptor and HER2 receptor). In particular embodiments, the tumor is a HER2 positive breast tumor or an estrogen receptor positive breast tumor, or a progesterone receptor breast tumor. In particular embodiments, the tumor is any form of breast tumor.

Also included are methods for modulating cancer signaling in a cell comprising administering to the cell a therapeutically effective amount of a therapeutic agent, wherein the therapeutic agent inhibits binding of one or more transcription factor to corresponding endogenous transcription factor binding sites, wherein the transcription factor(s) is (are) selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

### BRIEF DESCRIPTION OF THE DRAWINGS

FIG. 1A shows the average fold-change in cell confluency following treatment of oligonucleotide SEQ ID NO: 4593 in MDA-MB-231 triple negative breast cancer cells, as described in Example 1. Data normalized on the cell confluence for each tested condition measured at T=0, n=3. Treatment conditions: untreated cells, 0.12 uL RNAimax control, 0.5 uL RNAimax control, 50 nM of SEQ ID NO: 4593, and 100 nM of SEQ ID NO: 4593. *p<0.05, Student T test compared to untreated and corresponding RNAimax controls (0.12 uL for 50 nM, 0.5 uL for 100 nM). Data shown as mean±SEM.

FIG. 1B shows the average fold-change in cell confluency following treatment of oligonucleotide SEQ ID NO: 4593 in KPL4 her2+ breast cancer cells, as described in Example 1. Data normalized on the cell confluence for each tested condition measured at T=0, n=3. Treatment conditions: untreated cells, 0.12 uL RNAimax control, 0.5 uL, RNAimax control, 50 nM of SEQ ID NO: 4593, 100 nM of SEQ ID NO: 4593. *p<0.05, Student T test compared to untreated and corresponding RNAimax controls (0.12 uL for 50 nM, 0.5 uL for 100 nM). Data shown as mean±SEM.

FIG. 1C shows the average fold-change in cell confluency following treatment of oligonucleotide SEQ ID NO: 2225 in MDA-MB-231 triple negative breast cancer cells, as described in Example 1. Data normalized on the cell confluence for each tested condition measured at T=0, n=3. Treatment conditions: untreated cells, 0.12 uL RNAimax control, 0.5 uL RNAimax control, 50 nM of SEQ ID NO: 2225, and 100 nM of SEQ ID NO: 2225. *p<0.05, Student T test compared to untreated and corresponding RNAimax controls (0.12 uL for 50 nM, 0.5 uL for 100 nM). Data shown as mean±SEM.

FIG. 1D shows the average fold-change in cell confluency following treatment of oligonucleotide SEQ ID NO: 2225 in KPL4 her2+ breast cancer cells, as described in Example 1. Data normalized on the cell confluence for each tested condition measured at T=0, n=3. Treatment conditions: untreated cells, 0.12 uL RNAimax control, 0.5 uL RNAimax control, 50 nM of SEQ ID NO: 2225, 100 nM of SEQ ID NO: 2225. *p<0.05, Student T test compared to untreated and corresponding RNAimax controls (0.12 uL for 50 nM, 0.5 uL for 100 nM). Data shown as mean±SEM.

FIG. 2A shows the change in apoptotic cell count following treatment of oligonucleotide SEQ ID NO: 4593 in MDA-MB-231 breast cancer cells, as described in Example 2. Data normalized on cell confluence for each tested condition, n=3. *p<0.05, Student T test compared to untreated and corresponding RNAimax controls (0.12 uL for 50 nM, 0.5 uL for 100 nM), data shown as mean±SEM. Images of apoptotic cells labeled in red after treating with 100 nM of SEQ ID NO: 4593 at 24, 48, 72, 96 and 120 h after transfection are shown above the graph Treatment conditions: untreated cells, 0.12 uL RNAimax control, 0.5 uL. RNAimax control, 50 nM of SEQ ID NO: 4593, and 100 nM of SEQ ID NO: 4593.

FIG. 2B shows the change in apoptotic cell count following treatment of oligonucleotide SEQ ID NO: 2225 in MDA-MB-231 breast cancer cells, as described in Example 2. Data normalized on cell confluence for each tested condition, n=3. *p<0.05, Student T test compared to untreated and corresponding RNAimax controls (0.12 uL for 50 nM, 0.5 uL for 100 nM), data shown as mean±SEM.

FIG. 3A shows a representative fluorescent image of green fluorophore (ALEXA488) coupled oligonucleotide SEQ ID NO: 4593 localized in the nuclei of the MDA-MB-231 cells following transfection (detection indicated by white arrows), as described in Example 3.

FIG. 3B shows the average fold-change in cell confluency following treatment of the oligonucleotide (SEQ ID NO: 4593) in MDA-MB-231 cells, as described in Example 3. Treatment conditions: untreated cells, 0.12 uL RNAimax control (for 1, 25 and 50 nM of SEQ ID NO: 4593), 0.5 uL RNAimax control (for 100 and 200 nM of SEQ ID NO: 4593), 1 nM of SEQ ID NO: 4593, 10 nM of SEQ ID NO: 4593, 25 nM of SEQ ID NO: 4593, 50 nM of SEQ ID NO: 4593, 100 nM of SEQ ID NO: 4593, and 200 nM of SEQ ID NO: 4593.

FIG. 3C shows a dose response curve of the oligonucleotide (SEQ ID NO: 4593) in MDA-MB-231 cells. Efficacy was measured as a reduction in cell proliferation (area-under-the-curve of confluence curves) over 4 days starting 24h following transfection compared to corresponding RNAimax controls, as described in Example 3. Two Way ANOVA, p<0.001; n=3 per condition, exponential fit curve, R²>0.8.

FIG. 4A shows the average fold-change in cell confluency following treatment of the oligonucleotide (SEQ ID NOs: 4594, 4595, and 4596) alone or combined in MDA-MB-231 cells, as described in Example 4. The confluence over time of untreated cells and the effect of RNAImax alone (0.5 uL) are shown as controls. The effect of each sequence is statistically different from the effect of the combined sequences, Student T test, p<0.05. Data shown as mean±SEM.

FIG. 4B shows a dose response curve of the oligonucleotide (SEQ ID NOs: 4594, 4595, and 4596), as described in Example 4. MDA-MB-231 cells were transfected with 200 nM of SEQ ID NOs 4594, 4595, and 4596 or 50 nM of each of the sequences combined.

FIG. 5A shows the average fold-change in cell confluency over time following treatment of the oligonucleotide (SEQ ID NO: 88, 4597 and 4598) in MDA-MB-231 breast cancer cells, as described in Example 5. Treatment conditions: 100 nM of SEQ ID NOs 88, 4597, and 4598. Data normalized on cell confluence at T=0 for each tested condition and shown as mean±SEM The effects of SEQ ID NO: 4597, SEQ ID NO: 4598 and SEQ ID NO: 88 are statistically different from each other, Student T test, *p<0.05.

FIG. 5B shows the average fold-change in cell confluency over time following treatment of the oligonucleotide (SEQ ID NO: 88, 4597 and 4598) in KPL4 breast cancer cells, as described in Example 5. Treatment conditions: 100 nM of SEQ ID NOs 88, 4597, and 4598. The effect of SEQ ID NO: 88 is statistically different from the effects of SEQ ID NO: 4597 and of SEQ ID NO: 4598, Student T test, *p<0.05, NS=not statistically significant, n=3 per condition. Cells are transfected at T=0, data normalized on cell confluence at T=0 for each tested condition and shown as mean±SEM.

FIG. 6A shows the average fold-change in fraction of full length of the oligonucleotide (SEQ ID NOs: 88 and 4593) in human serum over time (in vitro incubation at 37° C.). Student T test, *p<0.05 compared to corresponding fraction of full length of SEQ ID NO: 88, data shown as mean±SEM.

FIG. 6B shows the average fold-change in cell confluency over time following transfection of the oligonucleotide (SEQ ID NOs: 88 and 4593), as described in Example 6. 50 nM, 100 nM and 200 nM of SEQ ID NO: 88 and SEQ ID NO: 4593 were tested. The effect of SEQ ID NO: 4593 is statistically different from the effect of SEQ ID NO: 88 at each tested concentration, Student T test, p<0.05, n=3 per condition, data shown as Mean±SEM.

FIGS. 7A-B show representative images of MDA-MB-231 cells 5 days after treatment with vehicle (FIG. 7A) and 100 nM of oligonucleotide SEQ: 88 (FIG. 7B).

FIG. 8 shows the average tumor volume (mm³) change over time following treatment with the oligonucleotide (SEQ ID NO: 4593) in MDA-MB231 human breast adenocarcinoma xenograft model in rat, as described in Example 8. Student T test, *p<0.05 compared to untreated.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

### Definitions

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight, or length that varies by as much as 30, 25, 20, 15, 10, 9, 8, 7, 6, 5, 4, 3, 2 or 1% to a reference quantity, level, value, number, frequency, percentage, dimension, size, amount, weight, or length.

“Binding,” as used in the context of transcription factors binding to therapeutic agents such as oligonucleotide decoys, refers to a direct interaction (e.g., non-covalent bonding between the transcription factor and the oligonucleotide decoy, including hydrogen-bonding, van der Waals bonding, etc.) between a transcription factor and an oligonucleotide decoy. Accordingly, a therapeutic agent such as an oligonucleotide that does not bind to a transcription factor does not directly interact with said transcription factor, and vice versa.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of:” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

“Homology” refers to the percentage number of nucleotides that are identical or constitute conservative substitutions. Homology may be determined using sequence comparison programs such as EMBOSS Pairwise Alignment Algorithm (available from the European Bioinformatics Institute (EBI)), the ClustalW program (also available from the European Bioinformatics Institute (EBI)), or the BLAST program (BLAST Manual, Altschul et al., Natl Cent. Biotechnol. Inf., Natl Lib. Med. (NCIB NLM NIH), Bethesda, Md., and Altschul et al., (1997) NAR 25:3389 3402), or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

The term “modulate” includes an “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more agents such as oligonucleotide decoys to produce or cause a greater physiological or cellular response in a cell or a subject, such as the activity of a transcription factor (e.g., gene expression), relative to the response caused by either no agent or a control compound. Relevant physiological or cellular responses (in vivo or in vitro) will be apparent to persons skilled in the art. An “increased” or “enhanced” amount or response may be “statistically significant” relative to an amount or response produced by no agent or a control composition, and may include an increase that is 1.1, 1.2, 2, 3, 4, 5, 6, 7, 8, 9, 10, 15, 20, 30, 40, 50 or more times (e.g., 500, 1000 times) (including all integers and ranges between and above 1, e.g., 1.5, 1.6, 1.7. 1.8) the amount or response produced by either no agent or a control compound. The term “reduce” or “inhibit” may relate generally to the ability of one or more agents such as oligonucleotide decoys to “decrease” a relevant pathological or physiological or cellular response in a cell or a subject, such as the activity of a transcription factor (e.g., gene expression), a pathological process (e.g., breast cancer signaling), or a symptom of a disease or condition described herein (e.g., breast cancer), relative to the response caused by either no agent or a control compound. Relevant physiological or cellular responses (in vivo or in vitro) will be apparent to persons skilled in the art and can be measured according to routine techniques. A “decrease” in a response may be “statistically significant” as compared to the response produced by no agent or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers and ranges in between.

“Modulation of gene expression level” includes any change in gene expression level, including an induction or activation (e.g., an increase in gene expression), an inhibition or suppression (e.g., a decrease in gene expression), or a stabilization (e.g., prevention of the up-regulation or down-regulation of a gene that ordinarily occurs in response to a stimulus, such as a cancer-inducing stimulus).

“Cancer signaling” refers to molecular and cellular mechanisms involved in the genesis (e.g., cell transformation), the growth (e.g., cell proliferation), the expansion and propagation (eg epithelial-mesenchymal transformation, cell migration or tissue invasion.

“Cancer” refers to Cancer is a generic term for a large group of diseases that can affect any part of the body. Other terms used are malignant tumours and neoplasms. One defining feature of cancer is the rapid creation of abnormal cells that grow beyond their usual boundaries, and which can then invade adjoining parts of the body and spread to other organs; the latter process is referred to as metastasis.

“Preventing” or “prevention” includes (1) a reduction in the risk of acquiring a disease or disorder (e.g., causing at least one of the clinical symptoms of a disease not to develop in a patient that may be exposed to or predisposed to the disease but does not yet experience or display symptoms of the disease), and/or (2) a reduction in the likely severity of a symptom associated with a disease or disorder (e.g., reducing the likely severity of at least one of the clinical symptoms of a disease in a patient that may be exposed to or predisposed to the disease but does not yet experience or display symptoms of the disease).

The terms “sequence identity” or, for example, comprising a “sequence 50% identical to,” as used herein, refer to the extent that sequences are identical on a nucleotide-by-nucleotide basis over a window of comparison. Thus, a “percentage of sequence identity” may be calculated by comparing two optimally aligned sequences over the window of comparison, determining the number of positions at which the identical nucleic acid base (e.g., A, T. C, or G) occurs in both sequences to yield the number of matched positions, dividing the number of matched positions by the total number of positions in the window of comparison (i.e., the window size), and multiplying the result by 100 to yield the percentage of sequence identity. In some embodiments, optimal alignment of sequences for aligning a comparison window may be conducted by using the EMBOSS Pairwise Alignment Algorithm (available from the European Bioinformatics Institute (EBI)), the ClustalW program (also available from the European Bioinformatics Institute (EBI)), or the BLAST program (BLAST Manual, Altschul et al., Natl Cent. Biotechnol. Inf., Natl Lib. Med. (NCIB NLM NIH), Bethesda, Md., and Altschul et al., (1997) NAR 25:3389 3402). In certain embodiments, the alignment of sequences for aligning a comparison window is conducted against the entire length of the reference sequence (e.g., from the Sequence Listing). In some embodiments, the alignment of sequences for aligning a comparison window is conducted against a portion of the reference sequence, for example, about, at least about, or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 contiguous nucleotides of the reference sequence.

A “subject” or a “subject in need thereof” or a “patient” includes a mammalian subject such as a primate or human subject.

“Treating” or “treatment” of any disease or disorder refers, in some embodiments, to ameliorating the disease or disorder (e.g., arresting or reducing the development of the disease or at least one of the clinical symptoms thereof). In some embodiments, “treating” or “treatment” refers to ameliorating at least one physical and/or biological parameter, which may not be discernible by the patient. In certain embodiments, “treating” or “treatment” refers to inhibiting the disease or disorder, either physically, (e.g., stabilization of a discernible symptom), physiologically, (e.g., stabilization of a physical parameter) or both. In some embodiments, “treating” or “treatment” refers to delaying the onset of the disease or disorder. “Treatment” or “prophylaxis” does not necessarily indicate complete eradication, cure, or prevention of the disease or condition, or associated symptoms thereof.

“Therapeutically effective amount” means the amount of a compound that, when administered to a patient, is sufficient to effect such treatment of a particular disease or condition. The “therapeutically effective amount” will vary depending on the compound, the disease, the severity of the disease, and the age, weight, etc., of the patient to be treated.

“Transcription factor network” means the network of regulatory interactions between transcription factors.

### Oligonucleotide Decoys and Other Therapeutic Agents

Embodiments of the present invention relate generally to therapeutic agents that inhibit binding of at least one transcription factor to at least one of its (endogenous) transcription binding site. Particular examples include oligonucleotide decoys that comprise one or more transcription binding sites that bind to at least one transcription factor, and thereby alter the ability of the transcription factor(s) to modulate gene expression. In embodiments, the oligonucleotide decoy binds to 1 to about 50 transcription factors, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 transcription factors, including any values or ranges thereof. In embodiments, the oligonucleotide decoy binds 1 to 40 transcription factors. In embodiments, the oligonucleotide decoy binds 2 to 40 transcription factors. In embodiments, the oligonucleotide decoy binds 2 to 30 transcription factors. In embodiments, the oligonucleotide decoy binds 2 to 20 transcription factors. In embodiments, the oligonucleotide decoy binds 2 to 10 transcription factors. In embodiments, the oligonucleotide decoy binds 2 to 5 transcription factors. In embodiments, the oligonucleotide decoy binds 18 to 22 transcription factors.

In embodiments, the oligonucleotide decoy comprises 1-50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, including any values or ranges therebetween) transcription factor binding sites that overlap. In embodiments, the oligonucleotide decoy comprises 2 to 40, 2 to 35, 2 to 30, 2 to 25, 2 to 20, 3 to 40, 3 to 35, 3 to 30, 3 to 25, or 3 to 20, including any values or ranges therebetween, transcription factor binding sites that overlap. In embodiments, the oligonucleotide decoy comprises 3 to 40 transcription factor binding sites that overlap. In embodiments, the oligonucleotide decoy comprises transcription factor binding sites that do not overlap.

In certain embodiments, the transcription factor is one or more members of following transcription factors: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

Thus, certain embodiments include an oligonucleotide decoy that comprises one or more (e.g., 1, 2, 3, 4, 5, etc.) transcription factor binding sites, where the one or more transcription factor binding site binds to a transcription factor selected from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

Also included are oligonucleotide decoys that comprise a combination of at least two (e.g., 2, 3, 4, 5, etc.) transcription factor binding sites, wherein each transcription factor binding site binds to a transcription factor selected from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors. Particular examples of combinations of transcription factor binding sites include those that bind to YBX1 and/or YY1, and/or ZEB2 and/or FOXM1 and/or FOXC1 and/or MAX and/or XBP1 and/or CXXC5 and/or NFIL3 and/or TCF4 and/or TCF7L1 and/or TGIF2 and/or MAF and/or LYL1 and/or GTF2IRD1 and/or E2F3 and/or E2F5 and/or BCL11A and/or HOXB5 and/or HMGA1.

The term “oligonucleotide” includes any double-stranded or substantially double-stranded, nucleic acid-containing polymer generally less than approximately 200 nucleotides (or 100 base pairs) and including, but not limited to, DNA, RNA and RNA-DNA hybrids with potential 5′ and/or 3′ overhangs.

In some embodiments, the oligonucleotide is about, at least about, or no more than about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 nucleotides in length (including all integers and ranges in between), and optionally comprises about, at least about, or no more than about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, or 200 base-paired nucleotides (including all integers and ranges in between). In embodiments, the oligonucleotide decoy is about 15 to about 60 base pairs in length. In embodiments, the oligonucleotide decoy is about 10 to about 100 base pairs in length. In embodiments, the oligonucleotide decoy is 12, 17, 18, 29, 41, 44, or 57-nucleotides in length.

In some embodiments, the oligonucleotide decoy comprises a first transcription factor binding site and a second or a third transcription up to 40 or more binding sites, optionally wherein the first transcription binding site and the second transcription binding site overlap and/or are in reverse orientation. In some embodiments, two double-stranded oligonucleotides overlap by at least 1-20 base pairs, for example, about 1, 2, 3, 4, 5, 6, 7, 8, 9, 10, 11, 12, 13, 14, 15, 16, 17, 18, 19, or 20 base pairs, including all values and ranges thereof. In some embodiments, the two double-stranded oligonucleotides overlap, and the two double-stranded oligonucleotides comprise 2, 3, 4, 5, 6, 7, 8, 9, or 10 consecutive identical base pairs. In specific embodiments, the first transcription factor binding site binds to a transcription factor selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

In some embodiments, the oligonucleotide decoy has flank sequences on its 5′ and/or 3 sides beyond transcription factor binding sites sequences. Flanks are short sequences, typically but not limited from 1 to 10 nucleotides that are added on the side of the sequences covered under this application. For instance, flanks to seq ID: 1 AACAAAGATTAA could be at/at and ccgc that could be added as follow to seq ID: 1 atatAACAAAGATTAAcgcg.

In certain embodiments, the oligonucleotide decoy (e.g., the sense strand of the decoy) comprises, consists, or consists essentially of a sequence (e.g., double-stranded sequence) from Table A.

Certain oligonucleotide decoys thus comprise, consist, or consist essentially of a sequence in Table A (e.g., SEQ ID NO: 1 to 48 for binding to TCF7L1 and GTF2IRD1, SEQ ID NO: 49 to SEQ ID NO: 64 for binding to FOXC1, FOXM1, GTF2IRD1 and TCF7L1, SEQ ID NO: 65 to SEQ ID NO: 176 for binding to E2F3, E2F5, FOXC1, FOXM1, GTF2IRD1, LYL1, TCF4, TCF7L1, TGIF2 and ZEB2, SEQ ID NO: 177 to SEQ ID NO: 3760 for binding to CXXC5, FOXC1, FOXM1, MAX, NFIL3, TCF4, XBP1, YBX1, YY1, ZEB2, SEQ ID NO: 3761 to SEQ ID NO: 3868 for binding to BCL11A, HMGA1, HOXB5 and MAF, SEQ ID NO: 3869 to SEQ ID NO: 4540 for binding to BCL11A, E2F3, E2F5, FOXC1, FOXM1, GTF2IRD1, HMGA1, HOXB5, LYL1, MAF, TCF4, TCF7L1, TGIF2 and ZEB2) or a variant of different size for the same transcription factor targets.

For instance, certain oligonucleotide decoys comprise contiguous or non-contiguous nucleotides of any of the sequences that bind to one or more transcription factors from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors. For non-contiguous portions, intervening nucleotides can be deleted or substituted with a different nucleotide, or intervening nucleotides can be added. Additional examples of variants include oligonucleotide decoys having at least about 70% sequence identity or homology (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology) to the entire length or a contiguous portion of a sequence.

In some embodiments, the contiguous portion is about, at least about, or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, or 100 contiguous nucleotides of a sequence.

An oligonucleotide decoy having a certain percent (e.g., 65%, 70%, 75%, 80%, 85%, 90%, 95%, or 99%) of sequence identity with another sequence means that, when aligned, that percentage determines the level of correspondence of bases arrangement in comparing the two sequences. This alignment and the percent homology or identity can be determined using any suitable software program known in the art that allows local alignment. In some embodiments, such programs include but are not limited to the EMBOSS Pairwise Alignment Algorithm (available from the European Bioinformatics Institute (EBI)), the ClustalW program (also available from the European Bioinformatics Institute (EBI)), or the BLAST program (BLAST Manual, Altschul et al., Natl Cent. Biotechnol. Inf., Natl Lib. Med. (NCIB NLM NIH), Bethesda, Md., and Altschul et al., (1997) NAR 25:3389 3402).

As noted above, one skilled in the art will recognize that the sequences encompassed by the invention include those that are fully or partially complementary to the sequences described herein, including those that hybridize under stringent hybridization conditions with an exemplified sequence. A nucleic acid is hybridizable to another nucleic acid when a single stranded form of the nucleic acid can anneal to the other single stranded nucleic acid under appropriate conditions of temperature and solution ionic strength. Hybridization conditions are well known in the art. In some embodiments, annealing may occur during a slow decrease of temperature from a denaturizing temperature (e.g., 100° C.) to room temperature in a water and/or salt containing solvent (e.g., Tris-EDTA buffer).

The population of oligonucleotide decoys can be composed of one oligonucleotide decoy, or a combination of two or more (e.g., 2, 3, 4, 5, etc.) oligonucleotide decoys. In certain embodiments, the population of oligonucleotide decoys is composed of one oligonucleotide decoy with a single transcription factor binding site from the list: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors. In some embodiments, the population of oligonucleotide decoys is composed of one oligonucleotide decoy with combination of at least two (e.g., 2, 3, 4, 5, etc.) transcription factor binding sites, which bind to factors from the list: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors. In some embodiments, the population of oligonucleotide decoys comprises one oligonucleotide decoy with combination of at least three (e.g., 3, 4, 5, etc.) transcription factor binding sites, which bind to the same or different (e.g., three or at least three different) factors from the list: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors. Other combinations will be apparent to persons skilled in the art.

In embodiments, the oligonucleotide decoy comprises a nucleotide sequence that is at least 80%, at least 85%, at least 90%, at least 95%, or 100% identical to a sequence selected from the group consisting of SEQ ID NO: 1 to SEQ ID NO: 48. In embodiments, the oligonucleotide decoy binds to one or more transcription factors selected from the group consisting of: TCF7L1 and GTF2IRD1. In embodiments, the present disclosure provides a nucleotide sequence that is at least 80%, at least 85%, at least 90%, at least 95%, or 100% identical to a sequence selected from the group consisting of SEQ ID NO: 49 to SEQ ID NO: 64. In embodiments, the oligonucleotide decoy binds to one or more transcription factors selected from the group consisting of: FOXC1, FOXM1, GTF2IRD1, and TCF7L1.

In embodiments, the present disclosure provides a nucleotide sequence that is at least 80%, at least 85%, at least 90%, at least 95%, or 100% identical to a sequence selected from the group consisting of SEQ ID NO: 3761 to SEQ ID NO: 3836. In embodiments, the oligonucleotide decoy binds to one or more transcription factors selected from the group consisting of: BCL11A, HMGA1, HOXB5, and MAF.

In embodiments, the present disclosure provides a nucleotide sequence that is at least 80%, at least 85%, at least 90%, at least 95%, or 100% identical to a sequence selected from the group consisting of SEQ ID NO: 3837 to SEQ ID NO: 3868. In embodiments, the oligonucleotide decoy binds to one or more transcription factors selected from the group consisting of: BCL11A, HMGA1, HOXB5, and MAF.

In embodiments, the present disclosure provides a nucleotide sequence that is at least 80%, at least 85%, at least 90%, at least 95%, or 100% identical to a sequence selected from the group consisting of SEQ ID SEQ ID NO: 65 to SEQ ID NO: 176, and SEQ ID NO: 4593. In embodiments, the oligonucleotide decoy binds to one or more transcription factors selected from the group consisting of: E2F3, E2F5, FOXC1, FOXM1, GTF2IRD1, LYL1, TCF4, TCF7L1, TGIF2, and ZEB2.

In embodiments, the present disclosure provides a nucleotide sequence that is at least 80%, at least 85%, at least 90%, at least 95%, or 100% identical to a sequence selected from the group consisting of SEQ ID SEQ ID NO: 177 to SEQ ID NO: 3760. In embodiments, the oligonucleotide decoy binds to one or more transcription factors selected from the group consisting of: CXXC5, FOXC1, FOXM1, MAX, NFIL3, TCF4, XBP1, YBX1, YY1, and ZEB2.

In embodiments, the present disclosure provides a nucleotide sequence that is at least 80%, at least 85%, at least 90%, at least 95%, or 100% identical to a sequence selected from the group consisting of SEQ ID SEQ ID NO: 3869 to SEQ ID NO: 4540. In embodiments, the oligonucleotide decoy binds to one or more transcription factors selected from the group consisting of: BCL11A, E2F3, E2F5, FOXC1, FOXM1, GTF2IRD1, HMGA1, HOXB5, LYL1, MAF, TCF4, TCF7L1, TGIF2, and ZEB2.

In embodiments, the oligonucleotide decoy binds to one or more transcription factors selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

In embodiments, the present disclosure provides a nucleotide sequence that is at least 80%, at least 85%, at least 90%, at least 95%, or 100% identical to a sequence selected from the group consisting of SEQ ID NO: 88, SEQ ID NO: 4593, SEQ ID NO: 4594, SEQ ID NO: 4595, SEQ ID NO: 4596, SEQ ID NO:4547, and SEQ ID NO: 4598.

In embodiments, the present disclosure provides a nucleotide sequence that is at least 80%, at least 85%, at least 90%, at least 95%, or 100% identical to a sequence selected from the group consisting of SEQ ID NO: 88, SEQ ID NO: 4593, SEQ ID NO: 4594, SEQ ID NO: 4595, SEQ ID NO: 4596, SEQ ID NO:4547, and SEQ ID NO: 4598.

Generally, the oligonucleotide decoys disclosed herein may be used to bind and, e.g., thereby inhibit, transcription factors that modulate the expression of genes involved cancer signaling. An oligonucleotide decoy that is designed to bind to a specific transcription factor has a nucleic acid sequence mimicking the endogenous genomic DNA sequence normally bound by the transcription factor. Accordingly, in some aspects the oligonucleotide decoys disclosed herein inhibit a necessary step for gene expression and regulation. Further, the oligonucleotide decoys disclosed herein may bind to one or a number of different transcription factors.

The term oligonucleotide encompasses sequences that include any of the known base analogs of DNA and RNA including, but not limited to, 2,6-diaminopurine, 5-carboxymethylaminomethyl-2-thiouracil, 5-carboxymethylaminomethyluracil, dihydrouracil, inosine, uracil-5-oxyacetic acid, N6-isopentenyladenine, 1-methyladenine, N-uracil-5-oxyacetic acid methylester, queosine, 2-thiocytosine, 5-bromouracil, methylphosphonate, phosphorodithioate, ormacetal, 3′-thioformacetal, nitroxide backbone, sulfone, sulfamate, morpholino derivatives, locked nucleic acid (LNA) derivatives, and/or peptide nucleic acid (PNA) derivatives. In some embodiments, the oligonucleotide is composed of two complementary single-stranded oligonucleotides that are annealed together. In some embodiments, the oligonucleotide is composed of one single-stranded oligonucleotide that forms intramolecular base pairs to create a substantially double-stranded structure.

In some embodiments, the oligonucleotide decoys disclosed herein are chemically modified by methods well known to the skilled artisan (e.g., incorporation of phosphorothioate, methylphosphonate, phosphorodithioate, phosphoramidates, carbonate, thioether, siloxane, acetamidate or carboxymethyl ester linkages between nucleotides), for example, to prevent degradation by nucleases within cells and/or in extra-cellular fluids (e.g., serum, cerebrospinal fluid). In some embodiments, the oligonucleotide decoys are designed to form hairpin and dumbbell structures, which can also prevent or hinder nuclease degradation. In particular embodiments, the oligonucleotide decoys are inserted as a portion of a larger plasmid capable of episomal maintenance or constitutive replication in the target cell in order to provide longer-term, enhanced intracellular exposure to the decoy sequence and/or reduce its degradation. Accordingly, any chemical modification or structural alteration known in the art to enhance oligonucleotide stability is within the scope of the present disclosure. In some embodiments, the oligonucleotide decoys disclosed herein may be attached, for example, to polyethylene glycol polymers, peptides (e.g., a protein translocation domain) or proteins which improve the therapeutic effect of oligonucleotide decoys. Such modified oligonucleotide decoys may preferentially traverse the cell membrane.

The oligonucleotide decoys described herein may generally be utilized as the free acid or free base. Alternatively, the oligonucleotide decoys may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids.

Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, calcium, potassium, magnesium, barium and calcium), as well as the ammonium ion and substituted derivatives thereof (e.g., dibenzylammonium, benzylammonium, 2-hydroxyethylammonium, and the like). Thus, the term “pharmaceutically acceptable salt” is intended to encompass any and all acceptable salt forms.

Prodrugs are also included. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the oligonucleotide decoys described herein. Further, in the case of a carboxylic acid (—COOH), esters may be employed, such as methyl esters, ethyl esters, and the like.

In certain embodiments, the oligonucleotide decoys are provided as salts, hydrates, solvates, or N-oxide derivatives. In certain embodiments, the oligonucleotide decoys are provided in solution (e.g., a saline solution having a physiologic pH) or in lyophilized form. In some embodiments, the oligonucleotide decoys are provided in liposomes.

The oligonucleotide decoys described herein may be made by conventional methods known in the art and thus are well within the knowledge of the skilled artisan. The activity of oligonucleotide decoys and variants thereof can be assayed according to routine techniques in the art (see the Examples). In particular embodiments, the oligonucleotide decoy is a synthetic oligonucleotide (i.e., a chemically-synthesized, non-naturally-occurring oligonucleotide).

Also included are non-oligonucleotide-based therapeutic agents, including those that inhibit binding of a transcription factor to its endogenous transcription binding site, for instance, by specifically binding to a KLF transcription factor, or by specifically binding to its endogenous transcription factor binding site (e.g., by mimicking the ZEB2 or FOXM1 transcription factor binding site). Examples of therapeutic agents include binding agents such as antibodies, small molecules, peptides, adnectins, anticalins, DARPins, anaphones, and aptamers, which exhibit binding specificity for a transcription factor from the list: BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors., e.g., a ZEB2 or FOXM1 factor transcription factor binding site domain, or which exhibit binding specificity for an endogenous ZEB2 or FOXM1 transcription factor binding site.

A binding agent is said to “exhibit binding specificity for,” “specifically bind to,” a transcription factor polypeptide (e.g., a transcription factor binding domain thereof), or an endogenous transcription factor binding site (e.g., double-stranded DNA sequence), if it reacts at a detectable level (within, for example, an ELISA or multiplex assay or an equivalent binding assay) with the polypeptide or nucleic acid, and does not react detectably in a significant (e.g., statistically significant) manner with unrelated structures under similar conditions.

The term “antibody” relates to an immunoglobulin whether natural or partly or wholly synthetically produced. The term also covers any polypeptide or protein having a binding domain which is, or is homologous to, an antigen-binding domain. CDR grafted antibodies are also contemplated by this term. The term “antigen-binding portion of an antibody,” “antigen-binding fragment,” “antigen-binding domain,” “antibody fragment” or a “functional fragment of an antibody” are used interchangeably in the present invention to include one or more fragments of an antibody that retain the ability to specifically bind to an antigen (see, e.g., Holliger et al., Nature Biotech. 23 (9): 1126-1129 (2005), which is hereby incorporated by reference in its entirety).

Antibodies may be prepared by any of a variety of techniques known to those of ordinary skill in the art. See, e.g., Harlow and Lane, Antibodies: A Laboratory Manual, Cold Spring Harbor Laboratory, 1988 which are hereby incorporated by reference in their entireties. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995 which are hereby incorporated by reference in their entireties. Particular examples include the VELOCIMMUNE® platform by REGENEREX® (see, e.g., U.S. Pat. No. 6,596,541 which is hereby incorporated by reference in its entirety) Antibodies can also be generated or identified by the use of phage display or yeast display libraries (see, e.g., U.S. Pat. No. 7,244,592; Chao et al., Nature Protocols. 1:755-768, 2006 which are hereby incorporated by reference in their entireties).

As noted above, “peptides” that inhibit binding of a transcription factor to its transcription factor binding site are included as binding agents. The term peptide typically refers to a polymer of amino acid residues and to variants and synthetic analogues of the same. In certain embodiments, the term “peptide” refers to relatively short polypeptides, including peptides that consist of about 3, 4, 5, 6, 7, 8, 9, 10, 11, 12, 13, 14, 15, 16, 17, 18, 19, 20, 25, 30, 31, 32, 33, 34, 35, 36, 37, 38, 39, 40, 41, 42, 43, 44, 45, 46, 47, 48, 49, 50 or more amino acids, including all integers and ranges (e.g., 5-10, 8-12, 10-15, 15-20, 20-25, 25-30, 30-40, 40-50) in between, and which, for example, bind to one or more regions of a KLF transcription factor, e.g., a transcription factor binding domain, or mimic the transcription factor by binding to at least one of its endogenous transcription factor binding sites. Peptides can be composed of naturally-occurring amino acids and/or non-naturally occurring amino acids.

As noted above, the present invention includes small molecules that inhibit binding of a transcription factor to its transcription factor binding site. A “small molecule” refers to an organic or inorganic compound that is of synthetic or biological origin, but is typically not a polymer. Organic compounds include a large class of chemical compounds whose molecules contain carbon, typically excluding those that contain only carbonates, simple oxides of carbon, or cyanides. A “polymer” refers generally to a large molecule or macromolecule composed of repeating structural units, which are typically connected by covalent chemical bond. In certain embodiments, a small molecule has a molecular weight of less than 1000-2000 Daltons, typically between about 300 and 700 Daltons, and including about 50, 100, 150, 200, 250, 300, 350, 400, 450, 500, 550, 500, 650, 600, 750, 700, 850, 800, 950, 1000 or 2000 Daltons.

Aptamers that inhibit binding of a transcription factor to its transcription factor binding site are also included as binding agents (see, e.g., Ellington et al., Nature. 346, 818-22, 1990; and Tuerk et al., Science. 249, 505-10, 1990, which are hereby incorporated by reference in their entireties). Examples of aptamers included nucleic acid aptamers (e.g., DNA aptamers, RNA aptamers) and peptide aptamers. Nucleic acid aptamers refer generally to nucleic acid species with secondary and tertiary structures that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620, which are hereby incorporated by reference in their entireties. Hence, included are nucleic acid aptamers that bind to one or more regions of a transcription factor, e.g., a transcription factor binding domain, or which bind to at least one of its endogenous transcription factor binding sites.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments may utilize the bacterial protein Thioredoxin-A as a scaffold protein, the variable loop being inserted within the reducing active site (-Cys-Gly-Pro-Cys- loop in the wild protein), with the two cysteine lateral chains being able to form a disulfide bridge. Methods for identifying peptide aptamers are described, for example, in U.S. Application No. 2003/0108532, which is hereby incorporated by reference in its entirety. Hence, included are peptide aptamers that bind to one or more regions of a transcription factor, e.g., a transcription factor binding domain, or which bind to at least one of its endogenous transcription factor binding sites. Peptide aptamer selection can be performed using different systems known in the art, including the yeast two-hybrid system.

| Also included are ADNECTINS™, AVIMERS™, and ANTICALINS that specifically bind to KLF transcription factor. ADNECTINS™ refer to a class of targeted biologics derived from human fibronectin, an abundant extracellular protein that naturally binds to other proteins. See, e.g., U.S. Application Nos. 2007/0082365; 2008/0139791; and 2008/0220049, which are hereby incorporated by reference in their entireties. ADNECTINS™ typically consists of a natural fibronectin backbone, as well as the multiple targeting domains of a specific portion of human fibronectin. The targeting domains can be engineered to enable an Adnectin™ to specifically recognize a therapeutic target of interest, such as a transcription factor polypeptide, or a fragment thereof, e.g., a transcription factor binding domain, or at least one of its endogenous transcription factor binding sites.

AVIMERS™ refer to multimeric binding proteins or peptides engineered using in vitro exon shuffling and phage display. Multiple binding domains are linked, resulting in greater affinity and specificity compared to single epitope immunoglobulin domains. See, e.g., Silverman et al., Nature Biotechnology. 23:1556-1561, 2005; U.S. Pat. No. 7,166,697; and U.S. Application Nos. 2004/0175756, 2005/0048512, 2005/0053973, 2005/0089932 and 2005/0221384, which are hereby incorporated by reference in their entireties.

Also included are designed ankyrin repeat proteins (DARPins), which include a class of non-immunoglobulin proteins that can offer advantages over antibodies for target binding in drug discovery and drug development. Among other uses, DARPins are ideally suited for in vivo imaging or delivery of toxins or other therapeutic payloads because of their favorable molecular properties, including small size and high stability. The low-cost production in bacteria and the rapid generation of many target-specific DARPins make the DARPin approach useful for drug discovery. Additionally, DARPins can be easily generated in multispecific formats, offering the potential to target an effector DARPin to a specific organ or to target multiple polypeptides/nucleic acids with one molecule composed of several DARPins. See, e.g., Stumpp et al., Curr Opin Drug Discov Devel. 10:153-159, 2007; U.S. Application No. 2009/0082274; and PCT/EP2001/10454, which are hereby incorporated by reference in their entireties.

Certain embodiments include “monobodies,” which typically utilize the 10th fibronectin type III domain of human fibronectin (FNfn10) as a scaffold to display multiple surface loops for target binding. FNfn10 is a small (94 residues) protein with a β-sandwich structure similar to the immunoglobulin fold. It is highly stable without disulfide bonds or metal ions, and it can be expressed in the correctly folded form at a high level in bacteria. The FNfn10 scaffold is compatible with virtually any display technologies. See, e.g., Batori et al., Protein Eng. 15:1015-20, 2002; and Wojcik et al., Nat Struct Mol Biol., 2010; and U.S. Pat. No. 6,673,901, which are hereby incorporated by reference in their entireties.

Anticalins refer to a class of antibody mimetics, which are typically synthesized from human lipocalins, a family of binding proteins with a hypervariable loop region supported by a structurally rigid framework. See, e.g., U.S. Application No. 2006/0058510, which is hereby incorporated by reference in its entirety. Anticalins typically have a size of about 20 kDa. Anticalins can be characterized by a barrel structure formed by eight antiparallel β-strands (a stable B-barrel scaffold) that are pairwise connected by four peptide loops and an attached a-helix. In certain aspects, conformational deviations to achieve specific binding are made in the hypervariable loop region(s). See, e.g., Skerra, FEBS J. 275:2677-83, 2008, herein incorporated by reference in its entirety.

The therapeutic agents, e.g. binding agents, described herein which inhibit the binding of a transcription factor comprised of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors to its endogenous transcription factor binding site(s), can be used in any of the methods and compositions described herein.

### Methods for Use

Embodiments of the present invention include methods of using therapeutic agents described herein (e.g., oligonucleotide decoys, binding agents), which inhibit or otherwise reduce binding of one or more transcription factors involved in breast cancer to its endogenous transcription binding site, and related compositions, to modulate the activity of one or more transcription factors involved in breast cancer. In particular embodiments, the one or more transcription factors is selected from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

The methods can be used, for example, to treat breast cancer in a subject, to modulate transcription of a gene present in a cell involved in breast cancer signaling, to modulate transcription of a gene present in a cell involved in breast cancer in a subject, and/or to modulate breast cancer signaling in a cell, for example, in a subject. Such methods can be practiced in vitro, for instance, by contacting a cell with a therapeutic agent (e.g., oligonucleotide decoy) or related composition, or in vivo, for instance, by administering to a subject in need thereof a therapeutic agent (e.g., oligonucleotide decoy) or related composition. In particular embodiments, the therapeutic agent is an oligonucleotide decoy or population of oligonucleotide decoys, as described herein.

Thus, certain embodiments include methods for treating breast cancer in a subject, comprising administering to the subject a therapeutically effective amount of a therapeutic agent, wherein the therapeutic agent inhibits binding of a transcription factor to its transcription binding site, and wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors. Also included are methods of treating breast cancer in a subject in need thereof, comprising administering to the subject a therapeutically effective amount of one or more oligonucleotide decoys described herein. In some embodiments, methods of preventing breast cancer in in a subject are provided, for example, prophylactic methods of treating or managing breast cancer. Such methods comprise administering to a subject in need thereof (e.g., a patient likely to develop breast cancer) a therapeutically effective amount of an oligonucleotide decoy described herein.

Thus, in certain embodiments, an oligonucleotide decoy and/or pharmaceutical composition comprising the same is administered to a subject in need thereof, for example, such as an animal (e.g., a bird, mammal, primate, human patient), suffering from or expected to suffer from breast cancer. In certain embodiments, the oligonucleotide decoys and/or pharmaceutical compositions thereof are administered to a patient, such as an animal, as a preventative measure against breast cancer including, but not limited to, any one or more of the possible forms of breast cancer. In some embodiments, the breast cancer is ER+, PR+, HER2+ or triple negative or combination of thereof (e.g., ER+/HER2+). In certain embodiments, the oligonucleotide decoys and/or pharmaceutical compositions thereof may be used for the prevention of one facet of breast cancer while concurrently treating another aspect of breast cancer.

In some embodiments, the therapeutic agent (e.g., oligonucleotide decoy, population of oligonucleotide decoys, binding agent) or composition that is administered to treat, manage, and/or prevent breast cancer binds to transcription factors selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of TCF7L1 and GTF2IRD1

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of TCF7L1, GTF2IRD1, FOXM1 and FOXC1.

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of TCF7L1, GTF2IRD1, FOXM1, FOXC1, ZEB2, TCF4, TGIF2, LYL1, E2F3 and E2F5.

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of TCF7L1, GTF2IRD1, FOXM1, FOXC1, ZEB2, TCF4, TGIF2, LYL1, E2F3, E2F5, MAF, BCL11A, HOXB5 and HMGA1.

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of YBX1, YY1, ZEB2, FOXM1, FOXC1, MAX, XBP1, CXXC5 and NFIL3.

In particular embodiments, the therapeutic agent (e.g., oligonucleotide decoy, population of oligonucleotide decoys, binding agent) or composition that is administered to treat, manage, and/or prevent breast cancer binds to transcription factors selected from the group consisting of YBX1, YY1, ZEB2, FOXM1, FOXC1, TCF7L1, TGIF2, LYL1, GTF2IRD1, E2F3 and E2F5.

Also included are methods for modulating transcription of a gene present in a cell involved in breast cancer signaling in a subject, comprising administering to the cell a therapeutically effective amount of a therapeutic agent, wherein the therapeutic agent inhibits binding of a transcription factor to its transcription factor binding site, wherein the transcription factor is selected from the group consisting of selected from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

In some embodiments, the therapeutic agent includes one or more oligonucleotide decoys described herein. In certain embodiments, modulation of transcription comprises suppressing or repressing gene expression. In some embodiments, modulation of transcription comprises stabilizing gene expression. In particular embodiments, modulation of transcription comprises activating or inducing gene expression. In certain embodiments, the gene is involved in breast cancer signaling. Genes involved in breast cancer signaling, but are not limited to, genes encoding DNA repair protein (e.g. nucleases), protein regulating gene expression (e.g., transcription factors, methylase), membrane proteins (e.g., ion channels, membrane receptors, etc.), soluble signaling molecules (e.g., intracellular signaling molecules or neurotransmitters), synthetic enzymes (e.g., neurotransmitter synthesis enzymes), and transcription factors.

Some embodiments include methods for modulating breast cancer signaling in a cell, comprising administering to the cell a therapeutically effective amount of a therapeutic agent, wherein the therapeutic agent inhibits binding of a transcription factor to its transcription factor binding site, wherein the transcription factor is selected from the group BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors. In some embodiments, the therapeutic agent includes one or more oligonucleotide decoys described herein. In certain embodiments, modulation of breast cancer signaling comprises suppressing or repressing breast cancer signaling. In some embodiments, modulation of breast cancer signaling comprises activation of an inhibitor of breast cancer signaling. In particular embodiments, modulation of breast cancer signaling comprises altering transcription factor networks.

In certain embodiments, the cell of the various methods is provided in vivo (e.g., in a subject suffering from breast cancer or likely to suffer from breast cancer). A cell provided in vivo can be located in different locations including breast but in a metastasis in another tissue, including but not limited to the skin, bones, lung or the brain. In other embodiments, the cell of the various methods is provided in vitro (e.g., in a petri dish). The cell can be any cell involved in breast cancer signaling, including, but not limited to, an epithelial cell, an endothelial cell, a duct cell or a stem cell (e.g., cancer stem cell), a tissue supportive cell (e.g., fibroblast), an immune cell, or a cell from a cell line (e.g., an MDA-MB-231 cell).

In some embodiments, the oligonucleotide decoys and/or pharmaceutical compositions thereof are used in combination therapy with at least one other therapeutic agent. Examples of other therapeutic agents include but are not limited to one or more additional oligonucleotide decoys or chemotherapy or immunotherapy. The oligonucleotide decoy and/or pharmaceutical composition thereof and the therapeutic agent can act additively or, more preferably, synergistically. In some embodiments, an oligonucleotide decoy and/or a pharmaceutical composition thereof is administered concurrently with the administration of another therapeutic agent, including another oligonucleotide decoy. In other embodiments, an oligonucleotide decoy or a pharmaceutical composition thereof is administered prior or subsequent to administration of another therapeutic agent, including another oligonucleotide decoy.

For administration to a subject in need thereof, the oligonucleotide decoys and/or pharmaceutical compositions described herein may be administered by any convenient route. Particular examples include administration by infusion or bolus injection in the tumor or intravenously, by absorption through epithelial or mucocutaneous linings (e.g., oral mucosa, rectal and intestinal mucosa, etc.), and by oral administration. Administration can be systemic or local. Various delivery systems are known in the art, including, e.g., encapsulation in liposomes, microparticles, microcapsules, capsules, etc., which can be used to administer a compound and/or pharmaceutical composition thereof. Methods of administration include, but are not limited to, intradermal, intramuscular, intraperitoneal, intravenous, subcutaneous, intranasal, epidural/peridural, oral, sublingual, intranasal, intracerebral, intravaginal, transdermal, rectally, by inhalation or topically, particularly to the ears, nose, eyes, or skin. In certain embodiments, the oligonucleotide decoy is administered perineurally, epidurally/peridurally, intrathecally, or intradermally. In certain embodiments, more than one oligonucleotide decoy is administered to a patient. The preferred mode of administration is left to the discretion of the practitioner, and will depend in-part upon the site of the medical condition.

In specific embodiments, it may be desirable to administer one or more oligonucleotide decoys locally to the area in need of treatment. This may be achieved, for example, and not by way of limitation, by local infusion during surgery, topical application (e.g., in conjunction with a wound dressing after surgery), by injection, by means of a catheter, by means of a suppository, or by means of an implant, said implant being of a porous, non-porous, or gelatinous material, including membranes, such as sialastic membranes, or fibers. In some embodiments, administration can be by direct injection at the site (e.g., former, current, or expected site) of breast cancer or of a metastasis.

In certain embodiments, it may be desirable to introduce one or more oligonucleotide decoys into the nervous system by any suitable route, including but not restricted to intraventricular, intrathecal, perineural and/or epidural/peridural injection Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

The amount of oligonucleotide decoy that will be effective in the treatment or prevention of breast in a patient will depend on the specific nature of the condition and can be determined by standard clinical techniques known in the art. In addition, in vitro or in vivo assays may optionally be employed to help identify optimal dosage ranges. The amount of an oligonucleotide decoy administered will, of course, be dependent on, among other factors, the subject being treated, the weight of the subject, the severity of the affliction, the manner of administration, and the judgment of the prescribing physician. In certain embodiments, a single dose of oligonucleotide decoy may comprise, but is not limited to, about 0.1 μg to 10,000 mg, 5 μg to about 1000 mg, about 50 μg to about 500 mg, about 100 μg to about 100 mg of oligonucleotide decoy per kilogram (kg) of body weight or per tumor mm² (i.e., size) or cm³ (i.e., volume). Frequency of administration can be once or multiple time daily, weekly, monthly or annually or any combination of those.

In some embodiments, the dosage forms are adapted to be administered to a patient as needed daily, once or more weekly, once or more monthly or once or more annually. Dosing may be provided alone or in combination with other drugs and may continue as long as required for effective treatment or prevention of breast cancer.

### Compositions and Kits

Certain embodiments include compositions, for example, pharmaceutical or therapeutic compositions, comprising one or more therapeutic agents (e.g., oligonucleotide decoys, binding agents) described herein, optionally in combination with one or more pharmaceutically-acceptable carriers (e.g., pharmaceutical-grade carriers).

The pharmaceutical compositions disclosed herein comprise a therapeutically effective amount of one or more therapeutic agents (e.g., oligonucleotide decoys), preferably, in purified form, together with a suitable amount of a pharmaceutically-acceptable carrier, so as to provide a form for proper administration to a patient. When administered to a patient, therapeutic agents such as oligonucleotide decoys and pharmaceutically-acceptable carriers are preferably sterile. Examples of pharmaceutically-acceptable carriers include, but are not limited to, saline, phosphate buffered saline (PBS), tris buffer, water, aqueous ethanol, emulsions, such as oil/water emulsions or triglyceride emulsions, tablets and capsules. Water is a preferred vehicle when oligonucleotide decoys are administered intravenously. Saline solutions and aqueous dextrose and glycerol solutions can also be employed as liquid vehicles, particularly for injectable solutions. Suitable pharmaceutically-acceptable carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds disclosed herein into preparations which can be used pharmaceutically. Proper formulation is dependent upon the route of administration chosen.

Pharmaceutical compositions can take the form of solutions, suspensions, emulsions, tablets, pills, pellets, capsules, capsules containing liquids, powders, sustained-release formulations, suppositories, aerosols, sprays, suspensions, or any other form suitable for use. Other examples of suitable pharmaceutical vehicles have been described in the art (see Remington's Pharmaceutical Sciences, Philadelphia College of Pharmacy and Science, 19th Edition, 1995, which is hereby incorporated by reference in its entirety).

Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol), oils, alcohols, slightly acidic buffers between pH 4 and pH 6 (e.g., acetate, citrate, or ascorbate at between about 5 mM to about 50 mM), etc. Additionally, flavoring agents, preservatives, coloring agents, bile salts, acylcarnitines and the like may be added.

For buccal administration, the compositions may take the form of tablets, lozenges, etc., formulated in conventional manner. Liquid drug formulations suitable for use with nebulizers and liquid spray devices and EHD aerosol devices will typically include a compound with a pharmaceutically acceptable vehicle. In some aspects, the pharmaceutically acceptable vehicle is a liquid such as alcohol, water, polyethylene glycol or a perfluorocarbon. Optionally, another material may be added to alter the aerosol properties of the solution or suspension of compounds. In some aspects, the material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611, which are hereby incorporated by reference in their entireties). A compound may also be formulated in rectal or vaginal compositions such as suppositories or retention enemas, e.g., containing conventional suppository bases such as cocoa butter or other glycerides. In addition to the formulations described previously, a compound may also be formulated as a depot preparation. Such long-acting formulations may be administered by implantation (for example, subcutaneously or intramuscularly) or by intramuscular injection. Thus, for example, a compound may be formulated with suitable polymeric or hydrophobic materials (for example, as an emulsion in an acceptable oil) or ion exchange resins, or as sparingly soluble derivatives, for example, as a sparingly soluble salt.

An oligonucleotide decoy may be included in any of the herein-described formulations, or in any other suitable formulation, as a pharmaceutically acceptable salt, a solvate or hydrate. Pharmaceutically acceptable salts substantially retain the activity of the parent compound and may be prepared by reaction with appropriate bases or acids and tend to be more soluble in aqueous and other protic solvents than the corresponding parent form.

In some instances, liposomes may be employed to facilitate uptake of the oligonucleotide decoys into cells, for example, in vitro or in a subject (see, e.g., Williams, S. A., Leukemia 10(12):1980-1989, 1996; Lappalainen et al., Antiviral Res. 23:119, 1994; Uhlmann et al., Chemical Reviews, Volume 90, No. 4, 25 pages 544-584, 1990; Gregoriadis, G., Chapter 14, Liposomes, Drug Carriers in Biology and Medicine, pp. 287-341, Academic Press, 1979, which are hereby incorporated by reference in their entireties). Hydrogels may also be used as vehicles for oligonucleotide decoy administration, for example, as described in WO 93/01286. Alternatively, the oligonucleotide decoys may be administered in microspheres or microparticles. (See, e.g., Wu, G. Y. and Wu, C. H., J. Biol. Chem. 262:4429-4432, 30 1987, which are hereby incorporated by reference in their entireties). Alternatively, the use of gas-filled microbubbles complexed with the oligonucleotide decoys can enhance delivery to target tissues, as described in U.S. Pat. No. 6,245,747, which is hereby incorporated by reference in its entirety. Sustained release compositions may also be used. These may include semipermeable polymeric matrices in the form of shaped articles such as films or microcapsules.

Oligonucleotide decoys can be introduced into cells using art-recognized techniques (e.g., transfection, electroporation, fusion, liposomes, colloidal polymeric particles and viral and non-viral vectors as well as other means known in the art). The method of delivery selected will depend at least on the oligonucleotide chemistry, the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, oligonucleotide decoys may be delivered using, e.g., methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (e. g., Dokka and Rojanasakul, Advanced Drug Delivery Reviews 44:35-49, incorporated by reference in its entirety).

In certain embodiments, one or more oligonucleotide decoys are provided in a kit. In certain embodiments, the kit includes an instruction, e.g., for using said one or more oligonucleotide decoys. In certain embodiments, said instruction describes one or more of the methods of the present invention, e.g., a method for preventing or treating breast cancer, a method of modulating gene expression in a cell, a method for modulating breast cancer signaling in a cell, a method for altering transcription factor networks in a cell, etc. In certain embodiments, the oligonucleotide decoys provided in a kit are provided in lyophilized form. In certain related embodiments, a kit that comprises one or more lyophilized oligonucleotide decoys further comprises a solution (e.g., a pharmaceutically-acceptable saline solution) that can be used to resuspend one or more of the oligonucleotide decoys.

### Embodiments

1. An oligonucleotide decoy, comprising: a combination of at least two transcription factor binding sites, wherein each transcription factor binding site binds to a transcription factor selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.
2. The oligonucleotide decoy of embodiment 1, wherein the oligonucleotide decoy is about 10 to about 100 base pairs in length.
3. The oligonucleotide decoy of embodiment 1, wherein the oligonucleotide decoy can bind from 2 transcription factors up to 30 or 40 or a higher number of transcription factors.
4. The oligonucleotide decoy of embodiment 1, wherein the oligonucleotide decoy comprises a first transcription factor binding site and a second transcription factor binding site, and wherein the first and the second transcription binding sites overlap.
5. The oligonucleotide decoy of embodiment 1, wherein the oligonucleotide decoy has a first transcription factor binding site, a second transcription factor binding site, and a third transcription factor binding site, and wherein the first, second, and third transcription factor binding sites overlap.
6. The oligonucleotide decoy of embodiment 1, wherein the oligonucleotide decoy has a first transcription factor binding site, a second transcription factor binding site, a third transcription factor binding site, and up to 30 or 40 or a higher number of transcription factor binding sites that can overlap.
7. The oligonucleotide decoy of embodiment 1, wherein the oligonucleotide decoy has transcription factor binding sites that do not overlap.
8. The oligonucleotide decoy of embodiment 1, which is any of the population of sequences that are 12 nucleotide long: SEQ ID NO: 1 to SEQ ID NO: 48.
9. The oligonucleotide decoy of embodiment 1, which is any of the population of sequences that are 17 nucleotide long: SEQ ID v: 49 to SEQ ID NO: 64.
10. The oligonucleotide decoy of embodiment 1, which is any of the population of sequences that are 18 nucleotide long: SEQ ID NO: 3761 to SEQ ID NO: 3836.
11. The oligonucleotide decoy of embodiment 1, which is any of the population of sequences that are 29 nucleotide long: SEQ ID NO: 3837 to SEQ ID NO: 3868.
12. The oligonucleotide decoy of embodiment 1, which is any of the population of sequences that are 41 nucleotide long: SEQ ID NO: 65 to SEQ ID NO: 176 and SEQ ID NO: 4593.
13. The oligonucleotide decoy of embodiment 1, which is any of the population of sequences that are 44 nucleotide long: SEQ ID NO: 177 to SEQ ID NO: 3760.
14. The oligonucleotide decoy of embodiment 1, which is any of the population of sequences that are 57 nucleotide long: SEQ ID NO: 3869 to SEQ ID NO: 4540.
15. The oligonucleotide decoy of embodiment 1, which is any of the population of possible sequences from the list of SEQ ID NO: 1 to SEQ ID NO: 48 that can bind to TCF7L1 and GTF2IRD1.
16. The oligonucleotide decoy of embodiment 1, which is any of the population of possible sequences from the list SEQ ID NO: 49 to SEQ ID NO: 64 that can bind to FOXC1, FOXM1, GTF2IRD1 and TCF7L1.
17. The oligonucleotide decoy of embodiment 1, which is any of the population of possible sequences from the list SEQ ID NO: 65 to SEQ ID NO: 176, and SEQ ID NO: 4593 that can bind to E2F3, E2F5, FOXC1, FOXM1, GTF2IRD1, LYL1, TCF4, TCF7L1, TGIF2 and ZEB2.
18. The oligonucleotide decoy of embodiment 1, which is any of the population of possible sequences from the list SEQ ID NO: 177 to SEQ ID NO: 3760 that can bind to CXXC5, FOXC1, FOXM1, MAX, NFIL3, TCF4, XBP1, YBX1, YY1, ZEB2.
19. The oligonucleotide decoy of embodiment 1, which is any of the population of possible sequences from the list SEQ ID NO: 3761 to SEQ ID NO: 3868 that can bind to BCL11A, HMGA1, HOXB5 and MAF.
20. The oligonucleotide decoy of embodiment 1, which is any of the population of possible sequences from the list SEQ ID NO: 3869 to SEQ ID NO: 4540 that can bind to BCL11A, E2F3, E2F5, FOXC1, FOXM1, GTF2IRD1, HMGA1, HOXB5, LYL1, MAF, TCF4, TCF7L1, TGIF2 and ZEB2.
21. The oligonucleotide decoy of embodiment 1, which is any of the population of possible sequences that can bind to any combination of transcription factor from the from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.
22. The oligonucleotide decoy of embodiment 1, wherein the oligonucleotide decoy comprises a nucleotide sequence of SEQ ID NO: 88, SEQ ID NO: 4593, SEQ ID NO: 4594, SEQ ID NO: 4595, SEQ ID NO: 4596, SEQ ID NO:4547, or SEQ ID NO: 4598.
23. The oligonucleotide decoy of embodiment 1, wherein the oligonucleotide decoy has a nucleotide sequence that is at least 80%, at least 90%, or at least 95% identical to a nucleotide sequence of SEQ ID NO: 88, SEQ ID NO: 4593, SEQ ID NO: 4594, SEQ ID NO: 4595, SEQ ID NO: 4596, SEQ ID NO:4547, or SEQ ID NO: 4598.
24. The oligonucleotide decoy of any one of embodiments 1-23, wherein the oligonucleotide decoy is a double-stranded nucleic acid.
25. A pharmaceutical composition, comprising: an oligonucleotide decoy or population of oligonucleotide decoy of any one of embodiments 1-24, and a pharmaceutically acceptable carrier.
26. A kit, comprising: an oligonucleotide decoy or population of oligonucleotide decoys of any one of embodiments 1-24, and optionally an instruction for using said oligonucleotide decoy.
27. A method for modulating transcription of a gene present in a cell involved in breast cancer signaling, comprising: administering to the cell an effective amount of an oligonucleotide decoy or population of oligonucleotide decoy of any one of embodiments 1-24.
28. A method for modulating breast cancer signaling in a cell, comprising: administering to the cell an effective amount of an oligonucleotide decoy or population of oligonucleotide decoy of any one of embodiments 1-24.
29. A method for treating breast cancer in a subject in need thereof, comprising: administering to the subject a therapeutically effective amount of an oligonucleotide decoy or population of oligonucleotide decoy of any one of embodiments 1-23.
30. The method of any one of embodiments 27 to 29, wherein the breast cancer is a triple negative breast cancer.
31. The method of any one of embodiments 27 to 29, wherein the breast cancer is an ER+ or PR+ breast cancer.
32. The method of any one of embodiments 27 to 29, wherein the breast cancer is an HER2+ breast cancer.
33. The method of any one of embodiments 27 to 29, wherein the breast cancer is any form of breast cancer.
34. A method for modulating breast cancer signaling in a cell, comprising: administering to the cell a therapeutically effective amount of a therapeutic agent, wherein the therapeutic agent inhibits binding of a transcription factor to its transcription factor binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.
35. A method for treating breast cancer in a subject in need thereof, comprising: administering to the subject a therapeutically effective amount of a therapeutic agent, wherein the therapeutic agent inhibits binding of a transcription factor to its transcription binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC. NFIL3, TCF (e.g., TCF3, TCF4, TCF7L1), TGIF2, XBP1, YBX1, YY1, ZEB2 and closely related factors.

### EXAMPLES

### Experimental Protocol and SEQ ID NOS

The invention may be further defined by reference to one or more of the following experimental protocols. It will be apparent to those skilled in the art that many modifications, both to materials and methods, may be practiced without departing from the scope of the invention.

### In Vitro Experiments

In one experiment, the effects of oligonucleotides binding to different combinations of the transcription factors from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors may be tested in cancer cell lines. The experimental cancer cell line models consist of testing the effects of oligonucleotides on fundamental properties of cancer cells in vitro: proliferation, which can impact tumor survival and growth and/or recurrence, and/or cell migration, which can impact tumor aggressiveness and/or metastatic potential.

In one instance, oligonucleotide of SEQ ID NO: 1 or SEQ ID NO: 17 or SEQ ID NO: 25 or SEQ ID NO: 33 binds to TCF7L1 and GTF2IRD1, oligonucleotide of SEQ ID NO: 49 binds to FOXC1, FOXM1, GTF2IRD1 and TCF7L1, oligonucleotide of SEQ ID NO: 65 or SEQ ID NO: 81 or SEQ ID NO: 97 or SEQ ID NO: 129 or SEQ ID NO: 161 binds to E2F3, E2F5, FOXC1, FOXM1, GTF2IRD1, LYL1, TCF4, TCF7L1, TGIF2, ZEB2.

In another instance, oligonucleotide of SEQ ID NO: 177 or SEQ ID NO: 433 or SEQ ID NO: 561 or SEQ ID NO: 817 or SEQ ID NO: 945 or SEQ ID NO: 1073 or SEQ ID NO: 1201 or SEQ ID NO: 1329 or SEQ ID NO: 1393 or SEQ ID NO: 1521 or SEQ ID NO: 1585 or SEQ ID NO: 1649 or SEQ ID NO: 1777 or SEQ ID NO: 1841 or SEQ ID NO: 1969 or SEQ ID NO: 2097 or SEQ ID NO: 2225 or SEQ ID NO: 2289 or SEQ ID NO: 2353 or SEQ ID NO: 2481 or SEQ ID NO: 2609 or SEQ ID NO: 2737 or SEQ ID NO: 2865 or SEQ ID NO: 2929 or SEQ ID NO: 2993 or SEQ ID NO: 3121 or SEQ ID NO: 3377 or SEQ ID NO: 3505 binds to CXXC5, FOXC1, FOXM1, MAX, NFIL3, TCF4, XBP1, YBX1, YY1, ZEB2.

In another instance, oligonucleotide of SEQ ID NO: 3761 or SEQ ID NO: 3785 or SEQ ID NO: 3791 or SEQ ID NO: 3795 or SEQ ID NO: 3799 or SEQ ID NO: 3805 or SEQ ID NO: 3809 or SEQ ID NO: 3813 or SEQ ID NO: 3837 or SEQ ID NO: 3853 binds to BCL11A, HMGA1, HOXB5, MAF.

In another instance, oligonucleotide of SEQ ID NO: 3869 or SEQ ID NO: 3901 or SEQ ID NO: 3965 or SEQ ID NO: 4157 binds to BCL11A, E2F3, E2F5, FOXC1, FOXM1, GTF2IRD1, HMGA1, HOXB5, LYL1, MAF, TCF4, TCF7L1, TGIF2, ZEB2.

Additional oligonucleotide sequences targeting the same combination of transcription factors or different combination of transcription factors from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors may be tested.

In one model, the effects of sequences, modified or not, targeting combinations of transcription factors from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors may be tested in the in vitro proliferation of cells from cancer cell lines representative of different types of breast cancers. Breast cancer cell lines in the model can be selected from the group consisting of, but not limited to: BT20, BT549, MDA-MB-157, MDA-MB-231, MDA-MB-436, MDA-MB-453, MDA-MB-468, MCF10A, Hs578, HBL100, SUM102, SUM149, MCF7, T47D, BT474, Hs578T, Hs587T, HCC1395, HCC1954, DT22, Dt28, MCF12A, HCC1954, T47D, HCC38, HCC38, HCC1187, HCC1150, HCC1937, HCC1954, CAMA1, ZR-75-1.

In one model, the effect of SEQ ID NOs: 1, 177, 3761 and 3869 (or related sequences), modified or not, on the in vitro proliferation of cells from cancer cell lines representative of different types of breast cancers (e.g., TBNC, ER+, HER2+) are tested. Breast cancer cell lines in the model can be selected from the group consisting of, but not limited to: BT20, BT549, MDA-MB-157, MDA-MB-231, MDA-MB-436, MDA-MB-453, MDA-MB-468, MCF10A, Hs578, HBL100, SUM102, SUM149, MCF7, T47D, BT474, Hs578T, Hs587T, HCC1395, HCC1954, DT22, Dt28, MCF12A, HCC1954, T47D, HCC38, HCC38, HCC1187, HCC1150, HCC1937, HCC1954, CAMA1, ZR-75-1.

In another model, the effect of sequences, modified or not, targeting combinations of transcription factors from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors may be tested in the in vitro migration of cells from cancer cell lines representative of different types of breast cancers. Breast cancer cell lines in the model can be selected from the group consisting of, but not limited to: BT20, BT549, MDA-MB-157, MDA-MB-231, MDA-MB-436, MDA-MB-453, MDA-MB-468, MCF10A, Hs578, HBL100, SUM102, SUM149, MCF7, T47D, BT474, Hs578T, Hs587T, HCC1395, HCC1954, DT22, Dt28, MCF12A, HCC1954, T47D, HCC38, HCC38, HCC1187, HCC1150, HCC1937, HCC1954, CAMA1, ZR-75-1.

In another model, the effect of SEQ ID NOs: 1, 177, 3761 and 3869 (or related sequences), modified or not, on the in vitro migration of cells from cancer cell lines representative of different types of breast cancers (e.g., TBNC, ER+, HER2+) are tested. Breast cancer cell lines in the model can be selected from the group consisting of, but not limited to: BT20, BT549, MDA-MB-157, MDA-MB-231, MDA-MB-436, MDA-MB-453, MDA-MB-468, MCF10A, Hs578, HBL100, SUM102, SUM149, MCF7, T47D, BT474, Hs578T, Hs587T, HCC1395, HCC1954, DT22, Dt28, MCF12A, HCC1954, T47D, HCC38, HCC38, HCC1187, HCC1150, HCC1937, HCC1954, CAMA1, ZR-75-1.

In the cell proliferation assays, cells are thawed from a frozen stock reserve, resuscitated and plated in wells 96 well plate in a standard growth media. Cells are treated with control vehicle or oligonucleotide sequences at up to 10 concentrations (e.g., 1, 10, 50, 100, 500, 1000, 2500, 5000, 10000 nM) using standard transfection method for cell lines (e.g., lipofectamine, DOTAP). The effect on treatments on cell numbers, which can increase, be stable or decrease, can be measured by a CellTiter-Glo (CTG) assay or an equivalent method at regular interval over a 5-10 day period.

In the cell migration assays, cells are thawed from a frozen stock reserve, resuscitated and plated in wells 96 well plate in a standard growth media. Cells are treated with control vehicle or oligonucleotide sequences at up to 10 concentrations (e.g., 1, 10, 50, 100, 500, 1000, 2500, 5000, 10000 nM) using standard transfection method for cell lines (e.g., lipofectamine, DOTAP). The effect on treatments on cell numbers, which can increase, be stable or decrease, can be measured by a Incucyte assay or an equivalent method at regular interval over a 5-10 day period.

### In Vivo Experiments

In one experiment, the effect of oligonucleotides binding to different combinations of the transcription factors from the group consisting of BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors may be tested in animal model of breast cancer. The experimental xenograft models consist of implanting human cancer cells, derived from human cell lines or from patients' primary tumors, in a mouse and test the effects of oligonucleotides on the tumor in an in vivo context. Models can be orthotopic when the cells are implanted in the breast fat pad of the animal, or heterotopic when implanted in another place, such as subcutaneously.

One model can include cell line derived xenografts from the cell lines tested in vitro listed above such as, but not limited to, MDA-MB-231 representing various breast cancer types and genetic and mutation backgrounds, including but not limited to triple negative triple negative, ER+ or HER2+ breast cancer cells.

Other models can include patient derived xenografts of the different types of breast cancers, including but not limited to triple negative, ER+ or HER2+ breast cancer. Oligonucleotides can be administered intravenously or directly in the tumor (intra-tumor) at different time point of the model. Oligonucleotides can also be administered to the cancer cells prior to in vivo implantation. Oligonucleotides can be administered once or multiple times during the course of the model. Animal weight, survival or tumor growth (e.g., volume and/or size) can be measured as endpoints to measure the effects of the oligonucleotides. Additional endpoints can be used.

### Oligonucleotides Annealing

Each pair of complementary strands of the oligonucleotide, at equimolar concentration, is annealed in water or in a saline buffer, possibly tris-EDTA (TE). The standard procedure includes either annealing at room temperature and/or maintaining the solution of both strands at a high denaturizing temperature for a period of time which may vary depending on the oligonucleotide pair, followed by a slow decrease in temperature until the solution reaches a low temperature of annealing. The proper annealing of complementary strands may be verified by any suitable standard technique, including but not restricted to running samples of annealed oligonucleotides next to un-annealed ones on a non-denaturing polyacrylamide gel.

### Oligonucleotide Binding

In one experiment, the binding of the oligonucleotides to transcription factors may be measured. In one instance, a commercial ELISA assay or a tailored version may be used using biotin-oligonucleotide probes incubated with nuclear protein extracts containing target transcription factors from cancer cell lines, and/or recombinant human transcription factors.

The processing of the decoy probe-protein mix may be performed according to the ELISA kit supplier: typically, the mix is loaded on streptavidin-coated 96-well plates, and the quantity of captured transcription factor can be measured with an antibody-based colorimetric in a microplate reader (OD₄₅₀ nm) following the protocol provided by the ELISA supplier.

In another experiment, the binding of the oligonucleotides to transcription factors may be measured with a multiplex assay or mesoscale assay or an equivalent assay.

### Transcription Factor Network Reverse Engineering and Analysis

In one instance, transcription factor networks may be reverse engineered from gene expression data from samples of normal and breast cancer tissue using network inference algorithms. Transcription factor networks may be compared together and the importance of individual transcription factor in those networks quantified using network topology or centrality “metrics to support the novel therapeutic potential of the transcription factors inhibited by oligonucleotides.

### Example 1: Effect of Oligonucleotide Decoys (SEQ ID NO: 4593 and SEQ ID NO: 2225) in Breast Cancer Cell Proliferation Suppression

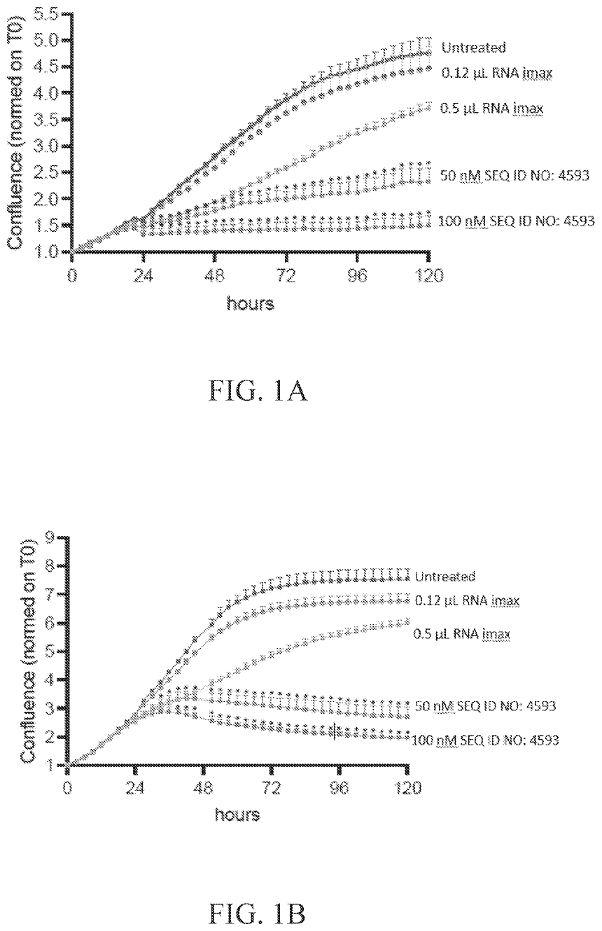

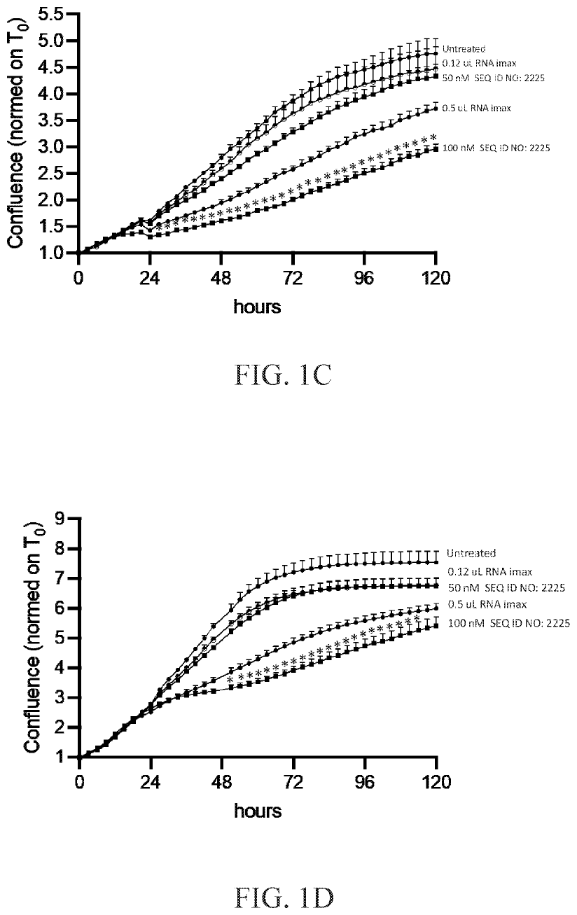

The oligonucleotide decoy SEQ ID NO: 4593 (modified SEQ ID NO: 88 with three 2′-methoxyethoxy bases at 5′ and 3′ ends on each strand) or SEQ ID NO: 2225 were transfected into MDA-MB-231 triple negative breast cancer cells and KPLA her2+ breast cancer cells. The average fold-change in cell confluency following treatment of the oligonucleotides was measured, as shown in FIGS. 1A-D. Cells were transfected with RNAimax (ThermoFisher) at T=0 and cell confluence was measured by Incucyte (Sartorius) every 3h up to 120h. Data was normalized on the cell confluence for each tested condition measured at T=0. *p<0.05, Student T test compared to untreated and corresponding RNAimax controls. Treatment conditions include: untreated, 0.12 uL RNAimax control, 0.5 uL RNAimax control, 50 nM of SEQ ID NO: 4593 or of SEQ ID NO: 2225, and 100 nM of SEQ ID NO. 4593 or of SEQ ID NO. 2225. As shown in FIGS. 1A and 1B, treating MDA-MB-231 triple negative breast cancer cells and KPL4 her2+ breast cancer cells with 50 nM or 100 nM of SEQ ID NO: 4593 significantly reduced the average cell confluency over time in both MDA-MB-231 and KPL4 breast cancer cells as compared to controls. As shown in FIGS. 1C and 1D, treating MDA-MB-231 triple negative breast cancer cells and KPL4 her2+ breast cancer cells with 100 nM of SEQ ID NO: 2225 significantly reduced the average cell confluency over time in both MDA-MB-231 and KPL4 breast cancer cells as compared to controls.

### Example 2: Apoptosis Induction of Oligonucleotide Decoys (SEQ ID NO: 4593 and SEQ ID NO: 2225) in Breast Cancer Cells

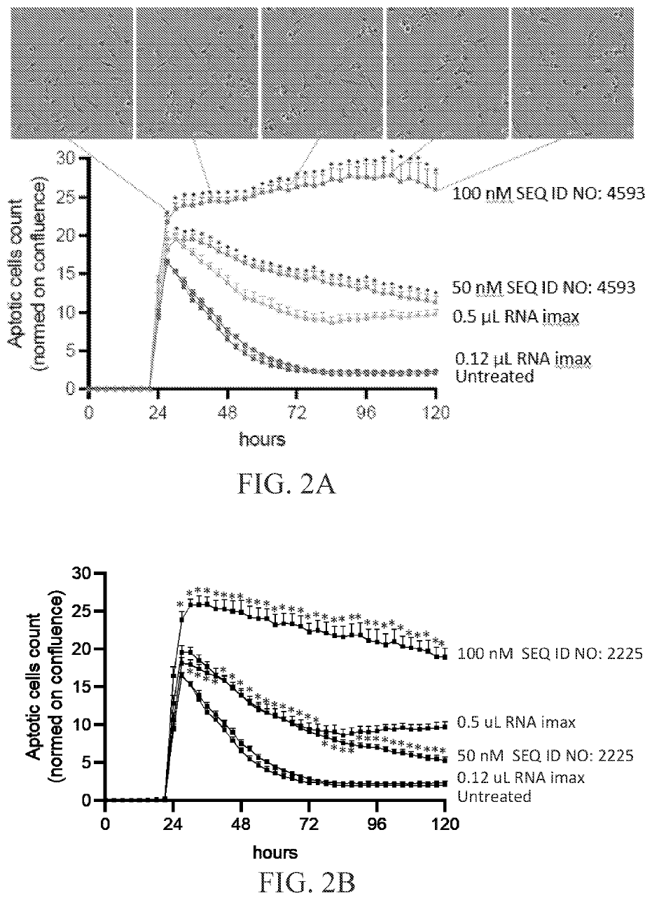

The effect of the oligonucleotide decoy of Example 1 (SEQ ID NO: 4593 and SEQ ID NO: 2225) on inducing MDA-MB-231 breast cancer cell apoptosis was evaluated. MDA-MB-231 breast cancer cells were transfected with RNAimax transfectant (ThermoFisher) at T=0. Apoptosis was measured using an Incucyte apparatus (Sartorius) with a substrate of caspase 3 and caspase 7 that emits a red fluorescence once catalyzed. The dye was applied 24 h after transfection. Data was normalized on cell confluence for each tested condition, n=3. *p<0.05, Student T test compared to untreated and corresponding RNAimax controls. Images of apoptotic cells labeled in red following treatment of 100 nM of SEQ ID NO: 4593 at 24, 48, 72, 96 and 120h after transfection were analyzed. As shown in FIG. 2A., both 50 nM and 100 nM of SEQ ID NO: 4593 treatment conditions significantly induced apoptosis in the breast cancer cells. As shown in FIG. 2B, both 50 nM and 100 nM of SEQ ID NO: 2225 treatment conditions significantly induced apoptosis in the breast cancer cells.

### Example 3: Dose Response of Oligonucleotide Decoy (SEQ ID NO: 4593) in Cancer Cell Proliferation Suppression

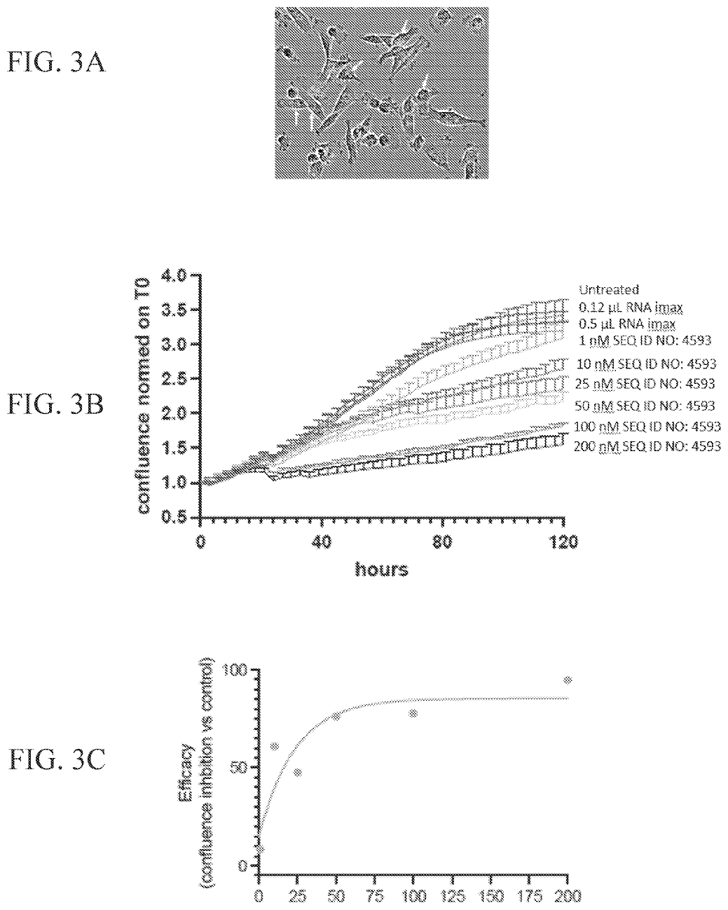

Oligonucleotide decoy SEQ ID NO: 4593 of Example 1 coupled to a green fluorophore (ALEXA488) in MDA-MB-231 cells were imaged following transfection. As shown in FIG. 3A, localization of the fluorophore-coupled oligonucleotide SEQ ID NO: 4593 in nuclei of the cells was observed. The effect of ascending doses of SEQ ID NO: 4593 on MDA-MB-231 cell proliferation was also assessed, as shown in FIG. 3B. Cells were transfected with RNAimax transfectant (ThermoFisher) at T=0. Treatment conditions included: untreated, 0.12 uL RNAimax control, 0.5 uL RNAimax control, 1 nM of SEQ ID NO: 4593, 10 nM of SEQ ID NO: 4593, 25 nM of SEQ ID NO: 4593, 50 nM of SEQ ID NO: 4593, 100 nM of SEQ ID NO: 4593, and 200 nM of SEQ ID NO: 4593. A dose response plot of SEQ ID NO: 4593 in MDA-MB-231 cells was obtained, as shown in FIG. 3C. Efficacy was measured as a reduction in cell proliferation (area-under-the-curve of confluence curves) over 4 days starting 24h following transfection compared to corresponding RNAimax controls.

### Example 4: Effect of Oligonucleotide Decoys (SEQ ID NOS: 4594, 4595, and 4596) in Breast Cancer Cell Proliferation Suppression

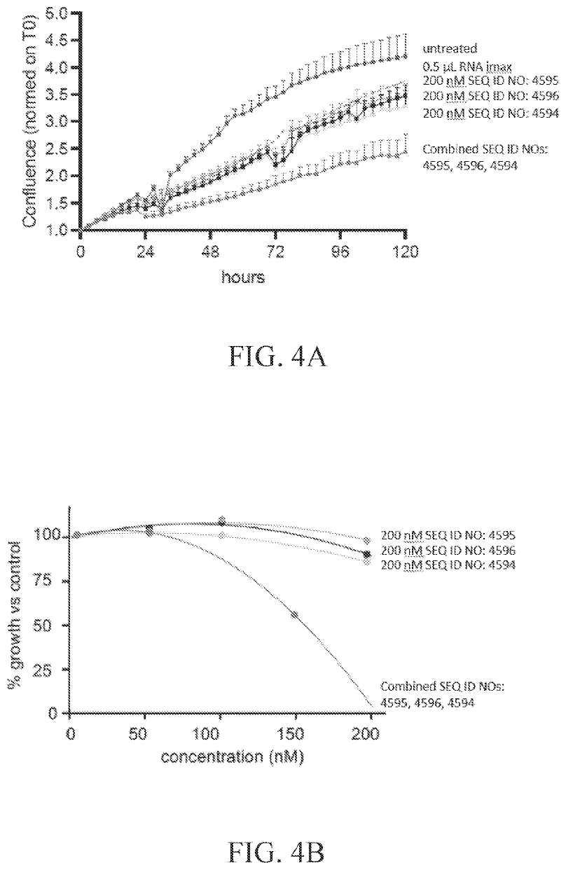

As shown in FIG. 4A, the effect of 200 nM oligonucleotide (SEQ ID NOs: 4594, 4595, and 4596) alone or combined (50 nM each of the sequences) in MDA-MB-231 cells was observed. SEQ ID NO: 4594 binds to FoxM1 and FoxC1 transcription factors, SEQ ID NO: 4595 binds to E2F3/E2F5, TCF7L1 and Lyl1 transcription factors, and SEQ ID NO: 4596 binds to TGIF2, GTFIRD1, ZEB2 and TCF4 transcription factors. The confluence values over time of untreated cells and the effect of RNAImax alone (0.5 uL) were used as controls. The effect of each sequence is statistically different from the effect of the combined sequences, Student T test, p<0.05. As shown in FIG. 4B, a dose response plot for SEQ ID NOs: 4594, 4595, 4596, and a combination of the sequences was obtained.

### Example 5: Effect of Oligonucleotide Decoys (SEQ ID NOS: 88, 4597, and 4598) in Breast Cancer Cell Proliferation Suppression

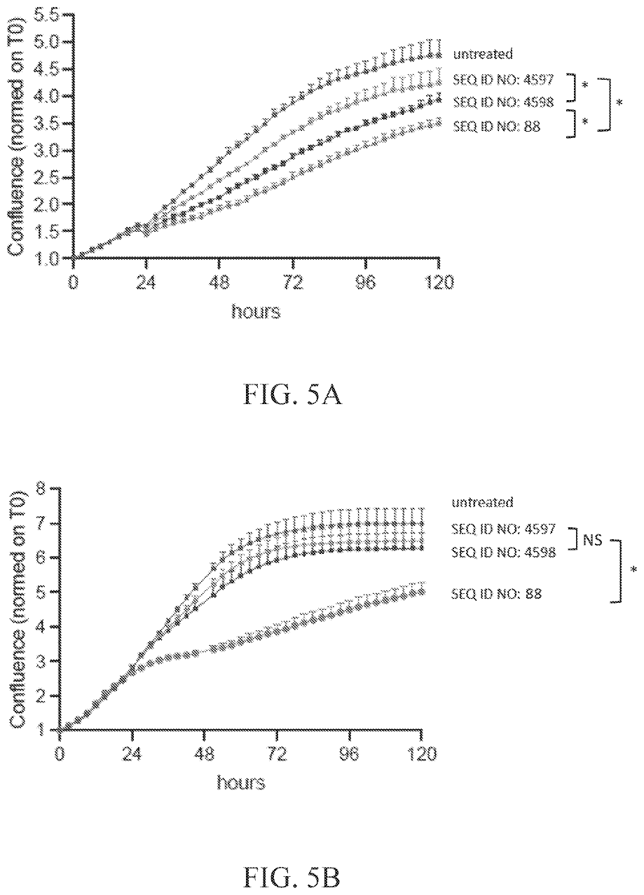

Cell proliferation suppression effect of SEQ ID NO: 88 and subparts of SEQ 4593 (i.e., SEQ ID NOs: 4597 and 4598) in two breast cancer cell lines, MDA-MB-231 cells and KPL4 cells, was assessed. SEQ ID NOs: 4597 and 4598 were generated from SEQ ID NO: 88, a short sequence binding to FOXM1, and a medium sequence binding to FOXM1, FOXC1, GTF2IRD1 and TCF7L1. The effect of each sequence on cell proliferation measured as confluence over time, as shown in FIGS. 5A and 5B. Cells were transfected at T=0. Data was normalized on cell confluence at T=0 for each tested condition and shown as mean±SEM. The effect of the short sequence SEQ ID NO: 4597 was statistically different from the effect of the medium sequence SEQ ID NO: 4598, and both SEQ ID NOs: 4597 and 4598 were different from the effect of the full sequence SEQ ID NO: 88. Student T test, *p<0.05, NS=not statistically significant, n=3 per condition, 100 nM for each sequence was used.

### Example 6: Stability and Efficacy of Oligonucleotide Decoys (SEQ ID NOs: 4593 and 88)

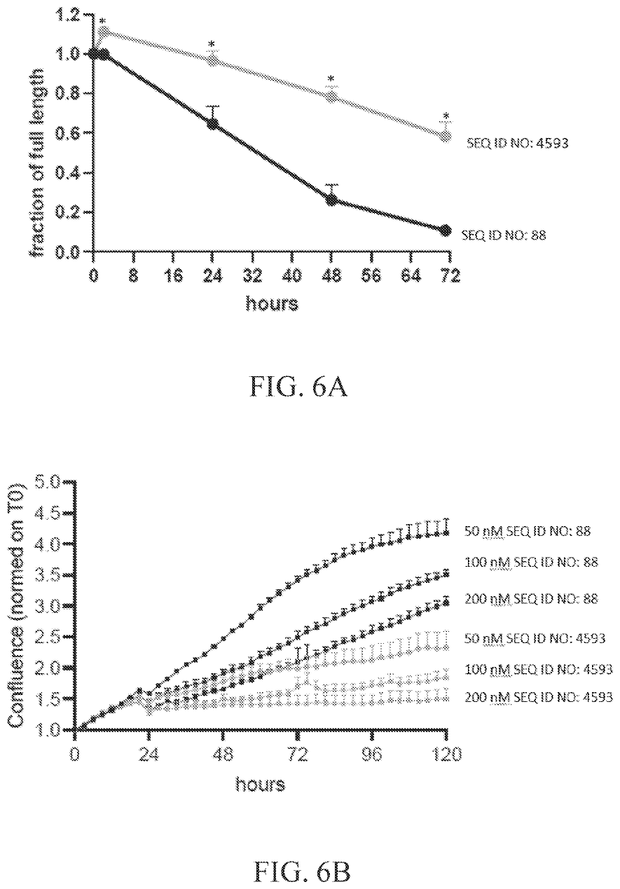

Stability of SEQ ID NOs 4593 and 88 was measured by observing the fold-change in fraction of full length of the oligonucleotides in human serum (in vitro incubation at 37° C.) over time. As shown in FIG. 6A, the 2′-methoxyethoxy-modified oligonucleotide (SEQ ID NO: 4593) exhibited more stability in serum as compared to the unmodified oligonucleotide (SEQ ID NO. 88) (half-life: ˜20 h for SEQ ID No: 88 versus ˜90 h for SEQ ID No: 4593), Student T test, *p<0.05 compared to corresponding fraction of full length of SEQ ID NO: 88. The efficacy of the unmodified and modified sequences for blocking MDA-MB-231 cell proliferation was measured as confluence over time, as shown in FIG. 6B. The cancer cell proliferation suppression effect of the modified sequence (SEQ ID NO: 4593) was higher than the effect of the unmodified sequence (SEQ ID NO: 88) at each dose level.

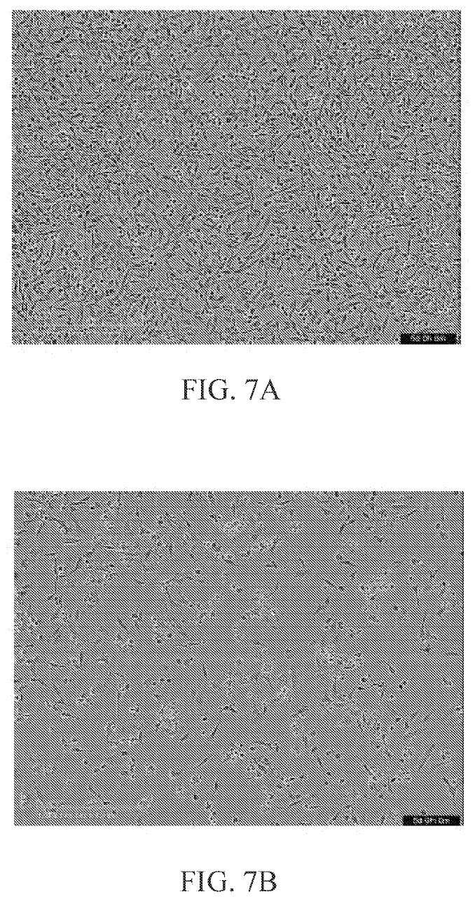

To study efficacy of SEQ ID NO: 88 in inducing apoptosis in breast cancer cells, images of MDA-MB-231 cells 5 days after treatment with vehicle (FIG. 7A) or SEQ ID NO: 88 (FIG. 7B) at 100 nM were obtained. 96 well plates were seeded with the same number of cells at T=0. Apoptosis was detected using a substrate of caspases 3 and 7 (sartorius) that emits a red fluorescence once catalyzed. As shown in FIGS. 7A-7B, SEQ ID NO: 88 treatment induced apoptosis in breast cancer cells.

### Example 8: Anti-Tumor Effects of Oligonucleotide Decoy (SEQ ID NO: 4593) in an MDA-MB231 (Triple Negative Breast Cancer Cells) Human Breast Adenocarcinoma Xenograft Model

Female Athymic Nude Rats (˜150 grams) were implanted subcutaneously with 1×10⁷ MDA-MB231 tumor cells in 50% Matrigel in their flanks. Study day 1 was determined when tumors reached a measurable size of ˜150 mm³ and rats were treated with the 2′-methoxyethoxy-modified oligonucleotide SEQ ID NO: 4593 and compared with untreated controls. SEQ ID NO: 4593 was intratumorally administered (ITU, 4.5 mg in a small 50 uL volume using the fan technique). Tumor growth was measured as the increase in growth in tumor volume (mm³) measured at regular intervals during the study up to Day 22. N=7-8 rats per group. *p<0.05, Student T test compared to untreated rats.

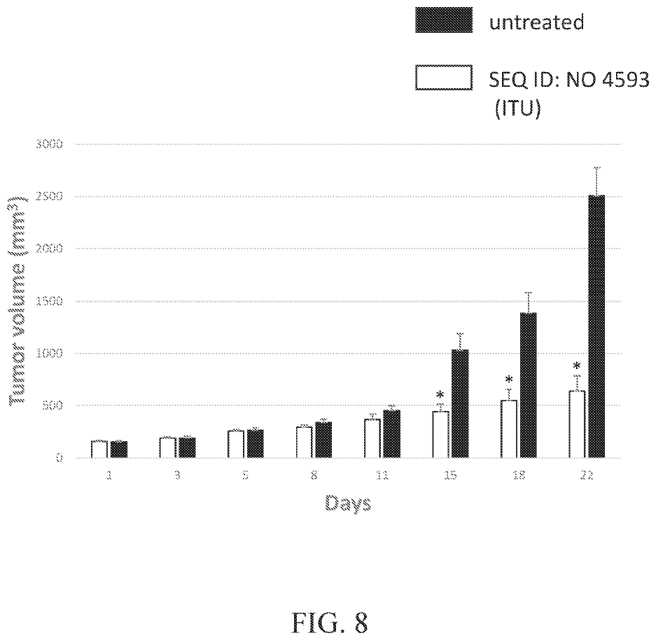

As shown in FIG. 8, SEQ ID NO: 4593 ITU treatment significantly inhibited tumor growth in rats. Animal growth rate measured by changes in body weight, clinical pathology (blood cells and serum chemistry), and histopathology of the liver and kidney indicated that SEQ ID NO: 4593 was well tolerated. When the oligonucleotide was intravenously administered, rat-specific rapid metabolism of the oligonucleotide was observed. The anti-tumor activity of SEQ ID NO: 4593 will be tested in other animal models.

### Example 9: Oligonucleotide Decoy Sequences

In the formulas and sequences described herein, “A” is an adenine nucleotide, “C” is a cytosine nucleotide, “G” is a guanine nucleotide, “T” is a thymine nucleotide, and “N” can be any nucleotide, preferably A, C, G, or T, “R” can be an A or G nucleotide, “Y” can be a C or T nucleotide, “S” can be a G or C nucleotide, “W” can be an A or T nucleotide, “K” can be a G or T nucleotide, “M” can be a A or C nucleotide, “B” can be a C or G or T nucleotide, “D” can be an A or G or T nucleotide, “H” can be an A or C or T nucleotide, “V” can be an A or C or G nucleotide. “inv” means inverted.

Although the formulas and sequences show a single strand, it should be understood that a complementary antisense strand is included as part of the structure of the oligonucleotide decoys. Some of exemplary sequences are listed below and summarized in Table A:

Formula 1:
(SEQ ID NO: 4541)
WWSAAAGATTAW
Formula 2:
(SEQ ID NO: 4542)
CTTTSWTAATCY
Formula 3:
(SEQ ID NO: 4543)
RGATTAWSAAAG
Formula 4:
(SEQ ID NO: 4544)
WTAATCTTTSWW
Formula 5:
(SEQ ID NO: 4545)
WWSAAAGTAAATAATCY
Formula 6:
(SEQ ID NO: 4546)
AACAGAAGGTAAACAAAGATTAWGGCGCCWWWTGACAGGTG
Formula 7:
(SEQ ID NO: 4547)
AACAGAAGGTAAACAAAGATTAWGGCGCCWWWTGACAGGTG
Formula 8:
(SEQ ID NO: 4548)
WWWWGGCGCCWTAATCTTTGTTTACCTTCTGTTGACAGGTG
Formula 9:
(SEQ ID NO: 4549)
CACCTGTCAACAGAAGGTAAACAAAGATTAWGGCGCCWWWW
Formula 10:
(SEQ ID NO: 4550)
CACCTGTCAWWWGGCGCCWTAATCTTTGTTTACCTTCTGTT
Formula 11:
(SEQ ID NO: 4551)
AARATGGCTCCACCTGTTTRTTTACACGTGAYRTAACANCGNTG
Formula 12:
(SEQ ID NO: 4552)
AARATGGCTCCACCTGTTTRTTTACACGTGTTAYRTCAYCGNTG
Formula 13:
(SEQ ID NO: 4553)
AARATGGCTCCACCTGTTAYRTCACGTGTAAAYAAACANCGNTG
Formula 14:
(SEQ ID NO: 4554)
AARATGGCTCCACCTGTTAYRTCAYCGNTGTTTRTTTACACGTG
Formula 15:
(SEQ ID NO: 4555)
AARATGGCTCCAYCGNTGTTTRTTTACACGTGAYRTAACACCTG
Formula 16:
(SEQ ID NO: 4556)
AARATGGCTCCAYCGNTGTTTRTTTACACGTGTTAYRTCACCTG
Formula 17:
(SEQ ID NO: 4557)
AARATGGCTCCAYCGNTGTTTRTTTACACGTGAYRTAACAGGTG
Formula 18:
(SEQ ID NO: 4558)
AARATGGCTCCAYCGRTGAYRTAACACCTGTTTRTTTACACGTG
Formula 19:
(SEQ ID NO: 4559)
AARATGGCTCCAYCGNTGTTAYRTCACCTGTTTRTTTACACGTG
Formula 20:
(SEQ ID NO: 4560)
AARATGGCTCCAYCGRTGAYRTAACAGGTGTTTRTTTACACGTG
Formula 21:
(SEQ ID NO: 4561)
AARATGGCTCCAYCGRTGAYRTAACACGTGTAAAYAAACACCTG
Formula 22:
(SEQ ID NO: 4562)
AARATGGCTCCAYCGNTGTTAYRTCACGTGTAAAYAAACACCTG
Formula 23:
(SEQ ID NO: 4563)
AARATGGCTCCAYCGRTGAYRTAACACGTGTAAAYAAACAGGTG
Formula 24:
(SEQ ID NO: 4564)
AARATGGCTCCAYCGNTGTTAYRTCACGTGTAAAYAAACAGGTG
Formula 25:
(SEQ ID NO: 4565)
CACCTGTTTRTTTACACGTGAYRTAACANCGRTGGAGCCATYTT
Formula 26:
(SEQ ID NO: 4566)
CAGGTGTTTRTTTACACGTGAYRTAACANCGRTGGAGCCATYTT
Formula 27:
(SEQ ID NO: 4567)
CACCTGTTTRTTTACACGTGTTAYRTCAYCGRTGGAGCCATYTT
Formula 28:
(SEQ ID NO: 4568)
CAGGTGTTTRTTTACACGTGTTAYRTCAYCGRTGGAGCCATYTT
Formula 29:
(SEQ ID NO: 4569)
CAGGTGAYRTAACACGTGTAAAYAAACANCGRTGGAGCCATYTT
Formula 30:
(SEQ ID NO: 4570)
CACCTGTTAYRTCACGTGTAAAYAAACANCGRTGGAGCCATYTT
Formula 31:
(SEQ ID NO: 4571)
CAGGTGTTAYRTCACGTGTAAAYAAACANCGRTGGAGCCATYTT
Formula 32:
(SEQ ID NO: 4572)
CACGTGTAAAYAAACAGGTGAYRTAACANCGRTGGAGCCATYTT
Formula 33:
(SEQ ID NO: 4573)
CACGTGTAAAYAAACACCTGTTAYRTCAYCGRTGGAGCCATYTT
Formula 34:
(SEQ ID NO: 4574)
CACGTGTAAAYAAACAGGTGTTAYRTCAYCGRTGGAGCCATYTT
Formula 35:
(SEQ ID NO: 4575)
CACGTGTAAAYAAACANCGRTGAYRTAACAGGTGGAGCCATYTT
Formula 36:
(SEQ ID NO: 4576)
CANCGNTGTTTRTTTACACGTGAYRTAACAGGTGGAGCCATYTT
Formula 37:
(SEQ ID NO: 4577)
CANCGRTGAYRTAACACGTGTAAAYAAACAGGTGGAGCCATYTT
Formula 38:
(SEQ ID NO: 4578)
CANCGNTGTTAYRTCACGTGTAAAYAAACAGGTGGAGCCATYTT
Formula 39:
(SEQ ID NO: 4579)
VGGYCATTAATWTCAGCR
Formula 40:
(SEQ ID NO: 4580)
VGGTCAGCAATTAATWTC
Formula 41:
(SEQ ID NO: 4581)
GAWATTAATGGGTCAGCR
Formula 42:
(SEQ ID NO: 4582)
GAWATTAATGRCCGCTGA
Formula 43:
(SEQ ID NO: 4583)
GAWATTAATTGCTGACCB
Formula 44:
(SEQ ID NO: 4584)
YGCTGACCCATTAATWTC
Formula 45:
(SEQ ID NO: 4585)
TCAGCGGYCATTAATWTC
Formula 46:
(SEQ ID NO: 4586)
YGCTGAWATTAATGRCCB
Formula 47:
(SEQ ID NO: 4587)
RGGAARTGAAATTAATTGCTGASTCAGCR
Formula 48:
(SEQ ID NO: 4588)
YGCTGASTCAGCAATTAATTTCAYTTCCY
Formula 49:
(SEQ ID NO: 4589)
AACAGAAGGTAAACAAAGATTAWGGCGCCWWWTGACAGGTGCTGACCCAT
TAATWTC
Formula 50:
(SEQ ID NO: 4590)
WWWWGGCGCCWTAATCTTTGTTTACCTTCTGTTGACAGGTGCTGACCCATT
AATWTC
Formula 51:
(SEQ ID NO: 4591)
AACAGAAGGTAAACAAAGATTAWGGCGCCWWWTGACAGGTGCTGAWATTA
ATGRCCB
Formula 52:
(SEQ ID NO: 4592)
WWWWGGCGCCWTAATCTTTGTTTACCTTCTGTTGACAGGTGCTGAWATTAA
TGRCCB

TABLE A

| SEQ ID NO | Sequence |
|---|---|
| 88 | AACAGAAGGT AAACAAAGAT TATGGCGCCT TTTGACAGGT G |
| 2225 | CACCTGTTTA TTTACACGTG TTACATCACC GATGGAGCCA TCTT |
| 4593 | A*A*C*AGAAGGT AAACAAAGAT TATGGCGCCT |
| | TTTGACAGG*T*G* |
| | * = 2′-methoxyethoxy |
| 4594 | GAAACAAACA AACAATCGTA AATAAACAG |
| 4595 | GATCAAAGGA ACAGATGGTT TTGGCGCCAA AAG |
| 4596 | GTTGACAGCT GTCAGGATTA ACGCACCTGC A |
| 4597 | ACAGAAGGTA AACAAA |
| 4598 | ACAGAAGGTA AACAAAGATT ATGGC |

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. An oligonucleotide decoy, comprising: at least two transcription factor binding sites, wherein each transcription factor binding site binds to a transcription factor selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

2. The oligonucleotide decoy of claim 1, wherein the oligonucleotide decoy is about 10 to about 100 base pairs in length.

3. The oligonucleotide decoy of claim 1, wherein the oligonucleotide decoy binds 2 to 40 transcription factors.

4. The oligonucleotide decoy of claim 1, wherein the oligonucleotide decoy comprises a first transcription factor binding site and a second transcription factor binding site, and wherein the first and the second transcription binding sites overlap.

5. The oligonucleotide decoy of claim 4, wherein the oligonucleotide decoy further comprises a third transcription factor binding site, wherein the first, second, and third transcription factor binding sites overlap.

6. The oligonucleotide decoy of claim 1, wherein the oligonucleotide decoy comprises 3-40 transcription factor binding sites that overlap.

7. The oligonucleotide decoy of claim 1, wherein the oligonucleotide decoy comprises transcription factor binding sites that do not overlap.

8. The oligonucleotide decoy of claim 1, wherein the oligonucleotide decoy is 12, 17, 18, 29, 41, 44, or 57-nucleotide long.

9. The oligonucleotide decoy of claim 1, comprising a nucleotide sequence that is at least 80%, at least 85%, at least 90%, at least 95%, or 100% identical to a sequence selected from the group consisting of SEQ ID NO: 1 to SEQ ID NO: 48.

10. The oligonucleotide decoy of claim 9, wherein the oligonucleotide decoy binds to one or more transcription factors selected from the group consisting of: TCF7L1 and GTF2IRD1.

11. The oligonucleotide decoy of claim 1, comprising a nucleotide sequence that is at least 80%, at least 85%, at least 90%, at least 95%, or 100% identical to a sequence selected from the group consisting of SEQ ID NO: 49 to SEQ ID NO: 64.

12. The oligonucleotide decoy of claim 11, wherein the oligonucleotide decoy binds to one or more transcription factors selected from the group consisting of: FOXC1, FOXM1, GTF2IRD1, and TCF7L1.

13. The oligonucleotide decoy of claim 1, comprising a nucleotide sequence that is at least 80%, at least 85%, at least 90%, at least 95%, or 100% identical to a sequence selected from the group consisting of SEQ ID NO: 3761 to SEQ ID NO: 3836.

14. The oligonucleotide decoy of claim 13, wherein the oligonucleotide decoy binds to one or more transcription factors selected from the group consisting of: BCL11A, HMGA1, HOXB5, and MAF.

15. The oligonucleotide decoy of claim 1, comprising a nucleotide sequence that is at least 80%, at least 85%, at least 90%, at least 95%, or 100% identical to a sequence selected from the group consisting of SEQ ID NO: 3837 to SEQ ID NO: 3868.

16. The oligonucleotide decoy of claim 15, wherein the oligonucleotide decoy binds to one or more transcription factors selected from the group consisting of: BCL11A, HMGA1, HOXB5, and MAF.

17. The oligonucleotide decoy of claim 1, comprising a nucleotide sequence that is at least 80%, at least 85%, at least 90%, at least 95%, or 100% identical to a sequence selected from the group consisting of SEQ ID NO: 65 to SEQ ID NO: 176, and SEQ ID NO: 4593.

18. The oligonucleotide decoy of claim 17, wherein the oligonucleotide decoy binds to one or more transcription factors selected from the group consisting of: E2F3, E2F5, FOXC1, FOXM1, GTF2IRD1, LYL1, TCF4, TCF7L1, TGIF2, and ZEB2.

19. The oligonucleotide decoy of claim 1, comprising a nucleotide sequence that is at least 80%, at least 85%, at least 90%, at least 95%, or 100% identical to a sequence selected from the group consisting of SEQ ID NO: 177 to SEQ ID NO: 3760.

20. The oligonucleotide decoy of claim 19, wherein the oligonucleotide decoy binds to one or more transcription factors selected from the group consisting of: CXXC5, FOXC1, FOXM1, MAX, NFIL3, TCF4, XBP1, YBX1, YY1, and ZEB2.

21. The oligonucleotide decoy of claim 1, comprising a nucleotide sequence that is at least 80%, at least 85%, at least 90%, at least 95%, or 100% identical to a sequence selected from the group consisting of SEQ ID NO: 3869 to SEQ ID NO: 4540.

22. The oligonucleotide decoy of claim 21, wherein the oligonucleotide decoy binds to one or more transcription factors selected from the group consisting of: BCL11A, E2F3, E2F5, FOXC1, FOXM1, GTF2IRD1, HMGA1, HOXB5, LYL1, MAF, TCF4, TCF7L1, TGIF2, and ZEB2.

23. The oligonucleotide decoy of claim 1, wherein the oligonucleotide decoy binds to one or more transcription factors selected from the group consisting of: BCL11A, CxxC domain-containing proteins (e.g., CXXC5), E2F (e.g., E2F5, E2F3), FOX (e.g., FOXC1, FOXM1), GTF2IRD1, HMGA1, HOXB5, LYL1, MAF (e.g., c-MAF, MAF-k), MAX, MYC, NFIL3, TCF (e.g., TCF3, TCF4, TCF7L1), TGIF2, XBP1, YBX1, YY1, ZEB2 and closely related factors.

24. The oligonucleotide decoy of claim 1, comprising a nucleotide sequence that is at least 80%, at least 85%, at least 90%, at least 95%, or 100% identical to a sequence selected from the group consisting of SEQ ID NO: 88, SEQ ID NO: 2225, SEQ ID NO: 4593, SEQ ID NO: 4594, SEQ ID NO: 4595, SEQ ID NO: 4596, SEQ ID NO:4547, and SEQ ID NO: 4598.

25. The oligonucleotide decoy of claim 1, comprising a nucleotide sequence that is at least 80%, at least 85%, at least 90%, at least 95%, or 100% identical to a sequence selected from the group consisting of SEQ ID NO: 88, SEQ ID NO: 4593, SEQ ID NO: 4594, SEQ ID NO: 4595, SEQ ID NO: 4596, SEQ ID NO:4547, and SEQ ID NO: 4598.

26. The oligonucleotide decoy of any one of claims 1-25, wherein the oligonucleotide decoy is a double-stranded nucleic acid.

27. A pharmaceutical composition, comprising:
an oligonucleotide decoy or population of oligonucleotide decoy of any one of claims 1-26, and
a pharmaceutically acceptable carrier.

28. A kit, comprising:
an oligonucleotide decoy or population of oligonucleotide decoys of any one of claims 1-26, and
optionally an instruction for using said oligonucleotide decoy.

29. A method for modulating transcription of a gene present in a cell involved in breast cancer signaling, comprising:
administering to the cell an effective amount of an oligonucleotide decoy of any one of claims 1-26.

30. A method for modulating breast cancer signaling in a cell, comprising:
administering to the cell an effective amount of an oligonucleotide decoy of any one of claims 1-26.

31. A method for treating breast cancer in a subject in need thereof, comprising:
administering to the subject a therapeutically effective amount of an oligonucleotide decoy of any one of claims 1-26.

32. The method of any one of claims 29 to 31, wherein the breast cancer is a triple negative breast cancer.

33. The method of any one of claims 29 to 31, wherein the breast cancer is an ER+ or PR+ breast cancer.

34. The method of any one of claims 29 to 31, wherein the breast cancer is an HER2+ breast cancer.

35. A method for modulating breast cancer signaling in a cell, comprising:
administering to the cell a therapeutically effective amount of a therapeutic agent, wherein the therapeutic agent inhibits binding of a transcription factor to its transcription factor binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.

36. A method for treating breast cancer in a subject in need thereof, comprising:
administering to the subject a therapeutically effective amount of a therapeutic agent, wherein the therapeutic agent inhibits binding of a transcription factor to its transcription binding site, wherein the transcription factor is selected from the group consisting of BCL11A, CxxC domain-containing proteins (e.g., CXXC5), E2F (e.g., E2F5, E2F3), FOX (e.g. FOXC1, FOXM1), GTF2IRD1, HMGA1, HOXB5, LYL1, MAF (e.g. c-MAF, MAF-k), MAX, MYC, NFIL3, TCF (e.g., TCF3, TCF4, TCF7L1), TGIF2, XBP1, YBX1, YY1, ZEB2 and closely related factors.
